(19) United States
(12) Patent Application Publication
(10) Pub. No.: US 2006/0074073 A1
(43) Pub. Date: Apr. 6, 2006

(54) Therapeutic combinations comprising poly (ADP-ribose) polymerases inhibitor

(75) Inventors: Heidi Marie Steinfeldt, San Diego, CA (US); Theodore James Boritzki, San Diego, CA (US); Alan Hilary Calvert, Blaydon-on-Tyne (GB); Nicola Jane Curtin, Rowlands Gill (GB); Mohamed Raza Dewji, Harrow (GB); Zdenek Hostomsky, La Jolla, CA (US); Christopher Jones, Gosforth (GB); Rhonda Kaufman, Bridgewater, NJ (US); Karen J. Klamerus, San Diego, CA (US); David Richard Newell, Humshaugh (GB); Elizabeth Ruth Plummer, Heddon-on-the-Wall (GB); Steven D. Reich, Del Mar, CA (US); Ian J. Stratford, Manchester (GB); Huw David Thomas, Houghton-le-Spring (GB); Kay Janine Williams, Middleton (GB)

Correspondence Address: AGOURON PHARMACEUTICALS, INC., 10777 SCIENCE CENTER DRIVE, SAN DIEGO, CA, 92121, US

(73) Assignees: AGOURON PHARMACEUTICALS, INC.; CANCER RESEARCH TECHNOLOGY LTD.

(21) Appl. No.: 11/231,470

(22) Filed: Sep. 20, 2005

Related U.S. Application Data
(60) Provisional application No. 60/612,458, filed on Sep. 22, 2004.
(60) Provisional application No. 60/683,006, filed on May 19, 2005.

(51) Int. Cl. A61K 31/55

(52) U.S. Cl. 514/212.06

(57) ABSTRACT

This invention generally relates to use of 8-fluoro-2{4-[(methylamino)methyl]phenyl}-1,3,4,5-tetrahydro-6H-azepino[5,4,3-cd]indol-6-one represented by formula 1 as a chemosensitizer that enhances the efficacy of cytotoxic drugs or radiotherapy. 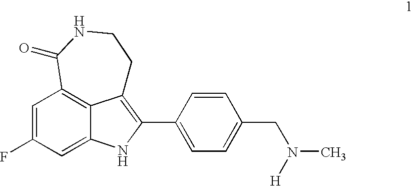 This invention provides pharmaceutical combinations of 8-fluoro-2{4-[(methylamino)methyl]phenyl}-1,3,4,5-tetrahydro-6H-azepino[5,4,3-cd]indol-6-one, or a pharmaceutically acceptable salt thereof and at least one additional therapeutic agent, kits containing such combinations and methods of using such combinations to treat subjects suffering from diseases such as cancer.

This application claims the benefit of U. S. Provisional Application No. 60/612,458 filed on Sep. 22, 2004, and U.S. Provisional Application No. 60/683,006 filed on May 19, 2005, the contents of which are hereby incorporated by reference in their entireties.

### FIELD OF THE INVENTION

This invention generally relates to use of 8-fluoro-2-{4-[(methylamino)methyl]phenyl}-1,3,4,5-tetrahydro-6H-azepino[5,4,3-cd]indol-6-one as a chemosensitizer that enhances the efficacy of cytotoxic drugs or radiotherapy. This invention provides pharmaceutical combinations of 8-fluoro-2-{4-[(methylamino)methyl]phenyl}-1,3,4,5-tetrahydro-6H-azepino[5,4,3-cd]indol-6-one, or a pharmaceutically acceptable salt thereof and at least one additional therapeutic agent, kits containing such combinations and methods of using such combinations to treat subjects suffering from diseases such as cancer.

### BACKGROUND OF THE INVENTION

The compound 8-fluoro-2-{4-[(methylamino)methyl]phenyl}-1,3,4,5-tetrahydro-6H-azepino[5,4,3-cd]indol-6-one represented by formula 1 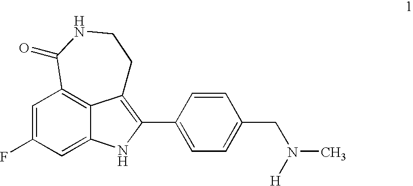
is a small molecule inhibitor of poly(ADP-ribose) polymerase (PARP). The compound of formula 1 and salts thereof, can be prepared as described in U.S. Pat. No. 6,495,541; PCT Application No. PCT/IB2004/000915, International Publication No. WO 2004/087713; U.S. Provisional Patent Application Nos. 60/612,457, 60/612,459 and 60/679,296, the disclosures of which are incorporated herein by reference in their entireties.

To date, eighteen enzymes have been identified by DNA sequence homology in the PARP family and the biochemical and enzymatic properties of seven have been investigated: PARP-1, and PARP-2 are stimulated by DNA strand breaks, PARP-3 interacts with PARP-1 and the centrosome, PARP-4 also known as vault PARP (VPARP) is the largest PARP and is associated with cytoplasmic vaults, tankyrase 1 and 2 (PARP-5a and 5b) are associated with telomeric proteins and the function of PARP-7 (TiPARP) is not clear at present but it may be involved in T-cell function and it can poly(ADP-ribosylate)histones (Ame JC, Splenlehauer C and de Murcia G. The PARP Superfamily. Bioassays 26 882-893 (2004)). Pharmacology studies have shown that the compound of formula 1 is an inhibitor of PARP-1 (Kᵢ=1.4 nM) and PARP-2 (Kᵢ=0.17 nM). Based on structural similarities in the amino acid sequences among the PARP enzymes, the compound of formula 1 likely binds with high affinity to the other members of the family as well.

Enzyme-mediated repair of single- or double-strand breaks in DNA is a potential mechanism of resistance to radiotherapy or cytotoxic drugs whose mechanism depends on DNA damage. Inhibition of DNA repair enzymes is thus a strategy for the potentiation of these agents. PARP-1, the best-characterized member of the PARP family, is a nuclear enzyme that upon activation by DNA damage mediates the transfer of ADP-ribose fragments from NAD⁺ to a number of acceptor proteins. Depending on the extent of DNA damage incurred, PARP-1 activation and subsequent poly(ADP-ribosyl)ation mediate the repair of the damaged DNA or induce cell death. When DNA damage is moderate, PARP-1 plays a significant role in the DNA repair process. Conversely, in the event of massive DNA damage, excessive activation of PARP-1 depletes ATP pools (in an effort to replenish NAD⁺), which ultimately leads to cell mortality by necrosis (Tentori L, Portarena I, Graziani G. Potential applications of poly(ADP-ribose) polymerase (PARP) inhibitors. Pharmacol Res 2002, 45, 73-85). This activation of PARP can also lead to release of AIF (apoptosis-inducing factor) triggering a caspase-independent apoptotic pathway. (Hong S J, Dawson T M and Dawson V L. Nuclear and mitochondrial conversations in cell death: PARP-1 and AIF. Trends in Pharmacological Sciences 25 259-264 (2004)).

As the result of the dual role of PARP-1, inhibitors of this enzyme, such as 8-fluoro-2-{4-[(methylamino)methyl]phenyl}-1,3,4,5-tetrahydro-6H-azepino[5,4,3-cd]indol-6-one represented by formula 1, may have a role as chemosensitizing agents (by preventing DNA repair, for example, after anticancer therapy), or as treatments for a variety of disease and toxic states that involve oxidative or nitric oxide induced stress and subsequent PARP hyperactivation. Such conditions include neurologic and neurodegenerative disorders (e.g., Parkinson's disease, Alzheimer's disease) (Love S, Barber R, Wilcock G K. Increased poly(ADP-ribosyl)ation of nuclear proteins in Alzheimer's disease. Brain 1999;122:247-53; Mandir A S, Przedborski S, Jackson-Lewis V, et al. Poly(ADP-ribose) polymerase activation mediates 1-methyl-4-phenyl-1,2,3,6-tetrahydropyridine (MPTP)-induced parkinsonism. Proc Natl Acad Sci USA 1999;96:5774-9); cardiovascular disorders (e.g., myocardial infarction, ischemia-reperfusion injury) (Pieper A A, Walles T, Wei G, et al. Myocardial postischemic injury is reduced by poly(ADP-ribose) polymerase-1 gene disruption. J Mol Med 2000;6:271-82; Szabó G, Bährle S, Stumpf N, et al. Poly(ADP-ribose)polymerase inhibition reduces reperfusion injury after heart transplantation. Circ Res 2002;90:100-6; U.S. Pat. No. 6,423,705); inflammatory diseases, (Szabó C, Dawson V. Role of poly(ADP-ribose) synthetase in inflammation and ischaemia-reperfusion. TIPS 1998;19:287-98); diabetic vascular dysfunction (Soriano F G, Virág L, Szabó C. Diabetic endothelial dysfunction: role of reactive oxygen and nitrogen species production and poly(ADP-ribose) polymerase activation. J Mol Med 2001;79:437-48); arthritis (Szabó C, Virág L, Cuzzocrea S, et al. Protection against peroxynitrite-induced fibroblast injury and arthritis development by inhibition of poly(ADP-ribose) synthase. Proc Natl Acad Sci USA 1998, vol.95, pp. 3867-72); and cisplatin-induced nephrotoxicity (Racz et al. “BGP-15—a novel poly(ADP-ribose) polymerase inhibitor—protects against nephrotoxicity of cisplatin without compromising its antitumor activity.” Biochem Pharmacol 2002;63:1099-111). Furthermore, it was shown that BRCA2 deficient tumor cells are acutely sensitive to PARP inhibitors alone (Bryant et al. “Specific killing of BRCA2 deficient tumors with inhibitors of poly(ADP-ribose)polymerase,” Nature, 2005, vol. 434, pp. 913-917; Farmer et al. “Targeting the DNA repair defect in BRCA mutant cells as a therapeutic strategy,” Nature, 2005, vol. 434, pp. 917-921). PARP inhibitors are also involved in enhancing the induction of the expression of Reg gene in β cells and HGF gene and, accordingly, promote the proliferation of pancreatic β-cells of Langerhans' islets and suppress apoptosis of the cells (U.S. Patent Application Publication 2004/0091453; PCT Publication No. WO 02/00665). In addition, PARP inhibitors are also used in cosmetic preparations, especially in after-sun lotions (PCT Publication No. WO 01/82877). There are no marketed PARP inhibitors presently.

Cancer remains a disease with high unmet medical need. Cytotoxic chemotherapy remains the mainstay of systemic therapy for the majority of cancers, particularly late-stage disease. However, for patients with advanced or metastatic disease, few of the cytotoxic chemotherapy agents or regimens have been effective in increasing overall survival. Furthermore, the small therapeutic window associated with cytotoxic agents results in significant toxicity in conjunction with suboptimal efficacy. Therefore, a chemosensitizer that enhances the efficacy of cytotoxic drugs at well-tolerated doses would fulfill a critical need for cancer patients.

Radiotherapy is an effective form of cancer treatment used in most tumor types for localized disease control. Over 50% of all cancer patients will receive radiotherapy during the course of their illness (Foroudi F. et al. An evidence-based estimate of appropriate radiotherapy utilization rate for breast cancer. Int J Radiat Oncol Biol Phys. 2002, 53:1240-53; Foroudi F. et al. An evidence-based estimate of the appropriate radiotherapy utilization rate for colorectal cancer. Int J Radiat Oncol Biol Phys. 2003, 56:1295-307; Foroudi F. et al. Evidence-based estimate of appropriate radiotherapy utilization rate for prostate cancer. Int J Radiat Oncol Biol Phys. 2003, 55:51-63; Barbera L. et al. Estimating the benefit and cost of radiotherapy for lung cancer. Int J Technol Assess Health Care. 2004, 20:545-51). However, even in front-line treatment of cancers, in which radiotherapy is administered with curative intent (for example, head and neck cancer, soft tissue sarcoma and carcinoma of the cervix), not all patients respond well. There is, therefore, a need for strategies that will enhance the overall patient response. Often standard chemotherapy will be administered prior to or post-radiotherapy. An alternative approach is to combine radiation treatment with novel anti-cancer agents that are specifically designed to enhance the efficacy of radiation treatment. Such agents impact upon the five key factors that govern tumor radiation response (“Cell survival as a determinant of tumor response.” Basic clinical radiobiology 3rd Edition. Steel G G (Ed.). Arnold Press UK, pp. 52-63, 2002). These are the capacity to repair the DNA-damage caused by radiation treatment; the redistribution of cells through the cell cycle following radiation treatment (such that tumor cells that were in a resistant phase at the first radiation dose may have progressed to a more sensitive phase by the next radiation fraction); repopulation, whereby surviving cells continue to divide thereby increasing the tumor burden between radiation fractions; reoxygenation of cells that survived the initial round of radiation treatment as a consequence of being more poorly oxygenated and finally, the inherent radiosensitivity of the particular tissue. Of these factors, enhanced repair and repopulation results in radioresistance whereas redistribution, reoxygenation and inherent radiosensitivity can render the tumor more responsive to radiation treatment. Clearly the use of agents that reduce the capacity for DNA-repair in combination with radiotherapy have potential to enhance radiotherapeutic outcome. PARP-1 activation and subsequent poly-(ADP-ribosylation) is seen in response to radiation-induced DNA-damage (Satoh M S & Lindahl T. “Role of poly(ADP-ribose) formation in DNA repair.” Nature. 1992, 356:356-358). Further, cell lines and knock-out mice generated to lack PARP-1 expression and activity show exquisite radiosensitivity supporting PARP-1 as an attractive target for radiopotentiation (Wang et al. “Mice lacking ADPRT and poly(ADP-ribosyl)ation develop normally but are susceptible to skin disease.” Genes Dev. 1995, 9:509-20; de Murcia et al. “Requirement of poly(ADP-ribose) polymerase in recovery from DNA damage in mice and in cells.” Proc Natl Acad Sci U S A. 1997, 94:7303-7; Masutani et al. “Function of poly(ADP-ribose) polymerase in response to DNA damage: gene-disruption study in mice.” Mol Cell Biochem. 1999, 193:149-52). In addition to direct affects on DNA-repair the class of PARP-1 inhibitors detailed are vasoactive and as such increase the potential for tumor reoxygenation between radiation fractions that can further contribute to enhanced radiation response (Calabrese et al. “Anticancer chemo- and radio-sensitisation in vitro and in vivo by a potent novel poly(ADP-ribose) polymerase-1 (PARP-1) inhibitor, AG14361.” J. Natl. Cancer Inst. 2004, 96: 56-67).

### SUMMARY OF THE INVENTION

In one embodiment, the present invention provides a dosage form for administration to a mammal, the dosage form comprising a compound of formula 1: 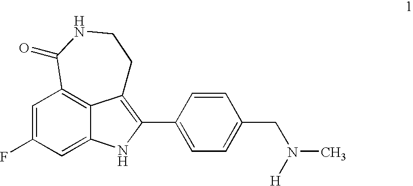
a pharmaceutically acceptable salt or solvate, or a mixture thereof, in an amount effective to provide a sustained plasma concentration value of at least 5.9 ng/mL of the compound of formula 1 for at least 24 hours after administration to the mammal.

In another embodiment, the invention provides a dosage form for administration to a mammal, the dosage form comprising a compound of formula 1, a pharmaceutically acceptable salt or solvate, or a mixture thereof, in an amount effective to provide a sustained plasma concentration value of at least 10 ng/mL of the compound of formula 1 for at least 24 hours after administration to the mammal.

In another embodiment, the invention provides a dosage form for administration to a mammal, the dosage form comprising a compound of formula 1, a pharmaceutically acceptable salt or solvate, or a mixture thereof, in an amount effective to provide a sustained plasma concentration value of at least 5.9 ng/mL of the compound of formula 1 for at least 24 hours after administration to the mammal, wherein the dosage form is a lyophilized powder for injection.

In another embodiment, the invention provides a dosage form for administration to a mammal, the dosage form comprising a compound of formula 1, a pharmaceutically acceptable salt or solvate, or a mixture thereof, in an amount effective to inhibit a poly(ADP-ribose) polymerase enzyme by at least 50% for at least 24 hours in peripheral blood lymphocytes after administration to the mammal.

In another embodiment, the invention provides a dosage form for administration to a mammal, the dosage form comprising a compound of formula 1, a pharmaceutically acceptable salt or solvate, or a mixture thereof, in an amount effective to inhibit a poly(ADP-ribose) polymerase enzyme by at least 50% for at least 24 hours in peripheral blood lymphocytes after administration to the mammal, wherein the dosage form is a lyophilized powder for injection.

In another embodiment, the invention provides a dosage form for administration to a mammal, the dosage form comprising a compound of formula 1, a pharmaceutically acceptable salt or solvate, or a mixture thereof, in an amount of from 1 to 48 mg/m² expressed as free base equivalent mass of the compound of formula 1.

In another embodiment, the invention provides a dosage form for administration to a mammal, the dosage form comprising a compound of formula 1, a pharmaceutically acceptable salt or solvate, or a mixture thereof, in an amount of from 1 to 48 mg/m² expressed as free base equivalent mass of the compound of formula 1, wherein the dosage form is a lyophilized powder for injection.

In another embodiment, the invention provides a dosage form for administration to a mammal, the dosage form comprising a compound of formula 1, a pharmaceutically acceptable salt or solvate, or a mixture thereof, in an amount of from 2 to 96 mg expressed as free base equivalent mass of the compound of formula 1.

In another embodiment, the invention provides a dosage form for administration to a mammal, the dosage form comprising a compound of formula 1, a pharmaceutically acceptable salt or solvate, or a mixture thereof, in an amount of from 2 to 96 mg expressed as free base equivalent mass of the compound of formula 1, wherein the dosage form is a lyophilized powder for injection.

In another embodiment, the invention provides a method of treating cancer in a mammal, the method comprising administering to the mammal

(a) a compound of formula 1, a pharmaceutically acceptable salt or solvate, or a mixture thereof in an amount effective to provide a sustained plasma concentration value of at least 5.9 ng/mL of the compound of formula 1 for at least 24 hours after administration to the mammal; and

(b) a therapeutically effective amount of at least one anti-cancer agent.

In another embodiment, the invention provides a method of treating cancer in a mammal, the method comprising administering to the mammal

(a) a compound of formula 1, a pharmaceutically acceptable salt or solvate, or a mixture thereof in an amount effective to provide a sustained plasma concentration value of at least 5.9 ng/mL of the compound of formula 1 for at least 24 hours after administration to the mammal; and

(b) a therapeutically effective amount of at least one anti-cancer agent, wherein the anti-cancer agent is administrated within 1 hour after administration of the compound of formula 1.

In another embodiment, the invention provides a method of treating cancer in a mammal, the method comprising administering to the mammal

(a) a compound of formula 1, a pharmaceutically acceptable salt or solvate, or a mixture thereof in an amount effective to provide a sustained plasma concentration value of at least 5.9 ng/mL of the compound of formula 1 for at least 24 hours after administration to the mammal; and

(b) a therapeutically effective amount of at least one anti-cancer agent, wherein the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In another embodiment, the invention provides a kit for treating cancer in a mammal, the kit comprising:

(a) an amount of a compound of formula 1, a pharmaceutically acceptable salt or solvate, or a mixture thereof, and a pharmaceutically acceptable carrier or diluent in a first unit dosage form;

(b) an amount of at least one anti-cancer agent and a pharmaceutically acceptable carrier or diluent in at least a second unit dosage form; and

(c) container for containing the first and at least the second dosage forms;

wherein the amount of the compound of formula 1 is effective to provide a sustained plasma concentration value of at least 5.9 ng/mL of the compound of formula 1 for at least 24 hours after administration to the mammal.

In another embodiment, the invention provides a method of treating cancer in a mammal, the method comprising administering to the mammal

(a) a compound of formula 1, a pharmaceutically acceptable salt or solvate, or a mixture thereof in an amount effective to provide a sustained plasma concentration value of at least 5.9 ng/mL of the compound of formula 1 for at least 24 hours after administration to the mammal; and

(b) a combination of irinotecan, 5-flourouracil and leucovorin.

In another embodiment, the invention provides a method of treating cancer in a mammal, the method comprising administering to the mammal

(a) a compound of formula 1, a pharmaceutically acceptable salt or solvate, or a mixture thereof in an amount effective to provide a sustained plasma concentration value of at least 5.9 ng/mL of the compound of formula 1 for at least 24 hours after administration to the mammal; and

(b) a dose of radiation effective to destroy the cancer.

Definitions and Abbreviations of Terms

The term “Compound I” refers to the phosphate salt of 8-fluoro-2-{4-[(methylamino)methyl]phenyl}-1,3,4,5-tetrahydro-6H-azepino[5,4,3-cd]indol-6-one. The term “the compound of formula 1” refers to 8-fluoro-2-4-[(methylamino)methyl]phenyl}-1,3,4,5-tetrahydro-6H-azepino[5,4,3-cd]indol-6-one, free base.

“Abnormal cell growth”, as used herein, unless otherwise indicated, refers to cell growth that is independent of normal regulatory mechanisms (e.g., loss of contact inhibition).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “radiosensitizer”, as used herein, means a drug that makes tumor cells more sensitive to radiation therapy.

The term “radiotherapy”, as used herein, includes external beam radiotherapy (XBRT) or teletherapy, brachytherapy or sealed source radiotherapy and unsealed source radiotherapy. The differences between these three main divisions of radiotherapy relate to the position of the radiation source; external is outside the body, while sealed and unsealed source radiotherapy has radioactive material delivered internally. External beam radiotherapy is the most common form of radiotherapy where a patient lies on a couch and an external source of X-rays is pointed at a particular part of the body. The radiation interacts with tissues and is absorbed, damaging the DNA of the cell. Brachytherapy is the delivery of radiation therapy using sealed sources which are placed as close as possible to the site to be treated. It is applicable for the treatment of tumors where a radiation source can be placed within a body cavity such as the oesophagus or bronchus or where the tumor is accessible to needle or catheter sources being placed within it, such as the head and neck and skin. Brachytherapy has potential applications to most tumor sites. It can be used as primary treatment or in combination with external beam radiotherapy. Unsealed source radiotherapy relates to the use of soluble forms of radioactive substances which are injected into the body. There is one common feature to all these substances, and that is the biological role of the non-radioactive parent substance. Proton therapy is a special case of external beam radiotherapy where the particles are protons.

The term “radio-immunotherapy”, as used herein, means radiotherapy where cytotoxic radionuclides are linked to antibodies in order to deliver toxins directly to tumor targets. Therapy with targeted radiation rather than antibody-targeted toxins (immunotoxins) has the advantage that adjacent tumor cells, which lack the appropriate antigenic determinants, can be destroyed by radiation cross-fire. Radioimmunotherapy is sometimes called targeted radiotherapy, but this latter term can also refer to radionuclides linked to non-immune molecules (radiotherapy).

The phrase “pharmaceutically acceptable salt(s)”, as used herein, unless otherwise indicated, includes salts of acidic or basic groups which may be present in a compound. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts. Particularly preferred salts include phosphate and gluconate salts.

The invention also includes isotopically-labeled compounds, which are identical to this recited in Formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F, and ³⁶Cl, respectively. Compounds of the present invention and pharmaceutically acceptable salts of said compounds, which contain the aforementioned isotopes and/or other isotopes of other atoms, are within the scope of this invention. Certain isotopically-labeled compounds of the present invention, for example those into which radioactive isotopes such as ³H, ¹⁴C, ¹¹C or ¹⁸F are incorporated, are useful in drug and/or substrate tissue distribution assays. Tritiated, i.e., ³H, and carbon-14, i.e., ¹⁴C, isotopes are particularly preferred for their ease of preparation and detectability and ¹¹C or ¹⁸F for use in positron emission tomography. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. An isotopically labeled compound of Formula 1 of this invention can generally be prepared by carrying out the procedures described for the non-labeled compound, substituting a readily available isotopically labeled reagent for a non-isotopically labeled reagent.

| | |
|---|---|
| ADP | adenosine diphosphate |
| AE | adverse event |
| ALT | alanine aminotransferase |
| ANC | absolute neutrophil count |
| AST | aspartate aminotransferase |
| AUC | area under the plasma concentration-time curve |
| AUC₍₀₋₂₄₎ | area under the plasma concentration-time |
| | curve from 0 to 24 hours |
| AUC₍₀₋ₜₗₐₛₜ₎ | area under the plasma concentration-time |
| | curve from time 0 to the last recorded observation |
| BLD | below limit of detection |
| BSA | Body surface area |
| BUN | blood urea nitrogen |
| C₀ | initial concentration |
| CL | clearance |
| Cₘₐₓ | maximum plasma concentration |
| CRC | colorectal cancer |
| CTCAEv3 | Common Terminology Criteria for Adverse Events |
| | version 3 |
| CV | cardiovascular |
| DLT | dose-limiting toxicities |
| DNA | deoxyribonucleic acid |
| EC₅₀ | concentration producing 50% of maximum effect |
| ECG | electrocardiogram |
| FcR | Fc receptor |
| 5-FU | 5-fluorouracil |
| GI | gastrointestinal |
| GIST | gastrointestinal stromal tumor |
| GLP | good laboratory practice |
| HCT | hematocrit |
| hERG | human ether-a-go-go-related gene |
| hERG-IKr | human ether-a-go-go-related gene channel blockade |
| HGB | hemoglobin |
| GI₅₀ | 50% cell growth inhibitory concentration |
| IC₅₀ | 50% enzyme activity inhibitory concentration |
| IGF | insulin-like growth factor |
| IGF-1R | insulin-like growth factor receptor, Type 1 |
| IL | interleukin |
| IP | intraperitoneal |
| IV | intravenous |
| LLN | lower limit of normal |
| LLOQ | lower limit of quantitation |
| LV | leucovorin |
| MMNG | N-methyl-N′-nitro-N-nitrosoguanidine |
| MTD | maximum tolerated dose |
| NAD | nicotinamide adenine dinucleotide |
| NOAEL | no-observed-adverse-effect level |
| PARP | poly(ADP-ribose) polymerase |
| PBMCs | peripheral blood monocytes |
| PD | pharmacodynamic |
| PID | PARP-inhibitory dose |
| PK | pharmacokinetic |
| PO | orally |
| RBC | red blood cells |
| RECIST | Response Evaluation Criteria in Solid Tumors |
| QC | Quality control |
| SAE | serious adverse event |
| SWFI/SWI | sterile water for injection |
| t_{1/2} | apparent terminal half-life |
| Tₘₐₓ | time of occurrence of Cₘₐₓ |
| ULN | upper limit of normal |
| Vdₛₛ | volume of distribution at steady-state |
| WFI | water for injection |

### BRIEF DESCRIPTION OF THE DRAWINGS

FIG. 1 represents the data on efficacy of temozolomide in combination with 8-fluoro-2-4-[(methylamino)methyl]phenyl}-1,3,4,5-tetrahydro-6H-azepino[5,4,3-cd]indol-6-one as the phosphate salt against the SW620 xenograft.

FIG. 2 represents the data on efficacy of temozolomide in combination with 8-fluoro-2-4-[(methylamino)methyl]phenyl}-1,3,4,5-tetrahydro-6H-azepino[5,4,3-cd]indol-6-one as the glucuronate salt against the SW620 xenograft.

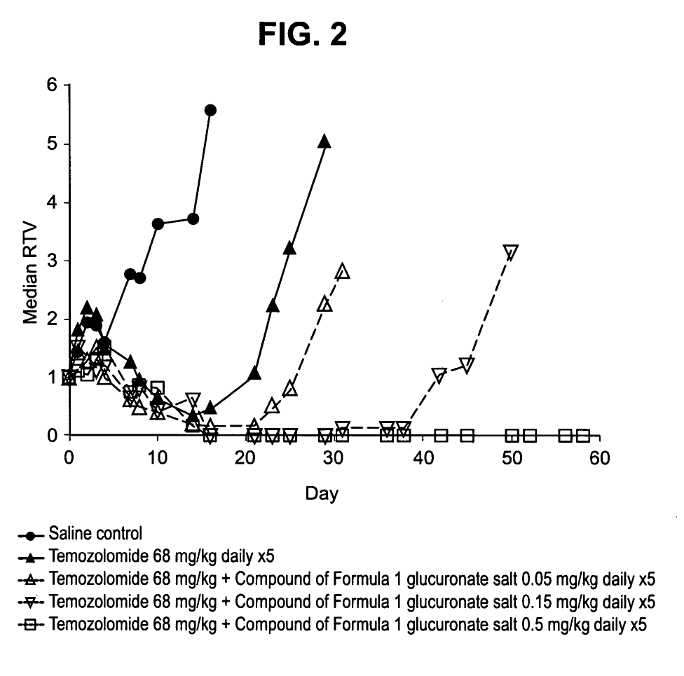

FIG. 3 represents the mean 8-fluoro-2-{4-[(methylamino)methyl]phenyl}-1,3,4,5-tetrahydro-6H-azepino[5,4,3-cd]indol-6-one plasma concentration-time profiles for Day -7 (the phosphate salt of 8-fluoro 2-{4-[(methylamino)methyl]phenyl}-1,3,4,5-tetrahydro-6H-azepino[5,4,3-cd]indol-6-one alone) a and 4 (the phosphate salt of 8-fluoro-2-{4-[(methylamino)methyl]phenyl}-1,3,4,5-tetrahydro-6H-azepino[5,4,3-cd]indol-6-one plus temozolomide) when the phosphate salt was given as a 30-minute IV Infusion and oral temozolomide was given as 100 mg/m².

FIG. 4 represents the Median PARP activity in peripheral blood lymphocytes following administration of the phosphate salt of 8-fluoro-2-{4-[(methylamino)methyl]phenyl}-1,3,4,5-tetrahydro-6H-azepino[5,4,3-cd]indol-6-one.

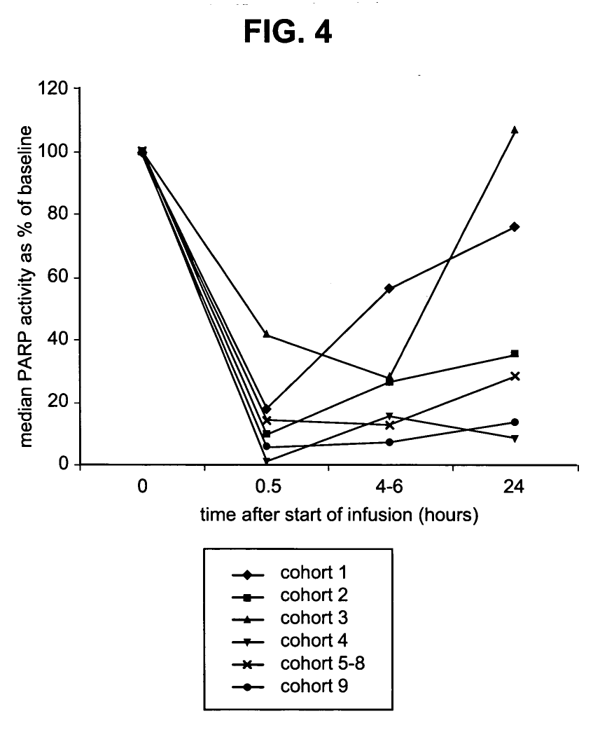

### DETAILED DESCRIPTION OF THE INVENTION

I. Pharmaceutical Formulations of 8-fluoro-2-{4-[(methylamino)methyl]phenyl}-1,3,4,5-tetrahydro-6H-azepino[5,4,3-cd]indol-6-one

The compound of formula 1 and salts thereof, can be prepared as described in U.S. Pat. No. 6,495,541; PCT application No. PCT/IB2004/000915; U.S. Provisional Patent Application No. 60/612,457; and U.S. Provisional Patent Application No. 60/612,459, the disclosures of which are incorporated herein by reference in their entireties. Certain starting materials may be prepared according to methods familiar to those skilled in the art and certain synthetic modifications may be done according to methods familiar to those skilled in the art.

The compound of formula 1 is capable of forming a wide variety of different salts with various inorganic and organic acids. Although such salts must be pharmaceutically acceptable for administration to mammals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid. Specific examples of preparation of a preferred salt, the phosphate salt, can be found in PCT application No. PCT/IB2004/000915; U.S. Provisional Patent Application No. 60/612,457; and U.S. Provisional Patent Application No. 60/612,459, the disclosures of which are incorporated herein by reference in their entireties.

Administration of the compound of formula 1 can be effected by any method that enables delivery of the compound to the site of action. These methods include oral routes, intraduodenal routes, parenteral injection (including intravenous, subcutaneous, intramuscular, intravascular or infusion), topical, and rectal administration.

The compound may, for example, be provided in a form suitable for oral administration as a tablet, capsule, pill, powder, sustained release formulation, solution, suspension, for parenteral injection as a sterile solution, suspension or emulsion, for topical administration as an ointment or cream or for rectal administration as a suppository.

The compound may be in unit dosage forms suitable for single administration of precise dosages. Preferably, dosage forms include a conventional pharmaceutical carrier or excipient and the compound of formula 1 as an active ingredient. In addition, dosage forms may include other medicinal or pharmaceutical agents, carriers, adjuvants, etc.

Exemplary parenteral administration forms include solutions or suspensions in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical composition may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials therefor include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

In preferred embodiments of the dosage forms of the invention, the dosage form is an oral dosage form, more preferably, a tablet or a capsule.

In preferred embodiments of the methods of the invention, the compound of formula 1 is parenterally administered, for example, using a lyophilized powder. Preparation of the lyophilized powder for injection for clinical use is described in U.S. Provisional Patent Application No. 60/612,459, the disclosure of which is incorporated herein by reference in its entirety.

For example, the phosphate salt of the compound of formula 1 may be formulated and supplied as a lyophilized powder for injection, 12 mg/vial (as free base), in 10 ml/20 mm, Type I, amber glass vials. The composition of the phosphate salt of the compound of formula 1 drug product may consist of the phosphate salt of the compound of formula 1, mannitol, water for injection, and nitrogen. The resulting drug product may be an off-white to yellow cake. Each drug product vial may be reconstituted with 6 mL sterile water for injection to yield a 2.02 mg/mL (rounded to 2 mg/mL), as free base of the compound of formula 1.

In preferred embodiments of the invention, plasma concentrations of the compound of formula 1 is maintained at or above 5.9 ng/mL. This value was determined from the target effect (IC89) for inhibition of cellular NAD⁺ depletion and poly-ADP-ribose polymer formation and adjusted for protein binding. Specifically, as shown in Example 4, the compound of formula 1 at 5 nM (temozolomide PF₅₀=1.3), greatly reduced the MNNG-induced cellular NAD⁺ consumption and inhibited cellular poly-ADP-ribose formation by 89% in A549 cells. Correcting the 5 nM target effect for human protein binding (27.4% mean unbound for the compound of formula 1 concentrations between 0.05 to 25 nM) yielded a plasma concentration of 5.9 ng/mL:

$$\frac{5 nM \times 323.37}{0.274 \times 1000} = 5.9 ng \text{/} mL$$

II. Pharmaceutical Combinations of the Present Invention and Their Use

In one embodiment of the present invention the compound of formula 1 is used to enhance the efficacy of cytotoxic drugs whose mechanism depends on DNA damage. These drugs include but not limited to temozolomide (SCHERING), irinotecan (PFIZER), topotecan (GLAXO SMITHKLINE), cisplatin (BRISTOL MEYERS SQUIBB; AM PHARM PARTNERS; BEDFORD; GENSIA SICOR PHARMS; PHARMACHEMIE), and doxorubicin hydrochloride (AM PHARM PARTNERS; BEDFORD; GENSIA; SICOR PHARMS; PHARMACHEMIE; ADRIA; ALZA).

Therapeutically effective amounts of the agents of the invention may be administered, typically in the form of a pharmaceutical composition, to treat diseases mediated by modulation or regulation of PARP. An “effective amount” is intended to mean that amount of an agent that, when administered to a mammal, including a human, in need of such treatment, is sufficient to effect treatment for a disease mediated by the activity of one or more PARP enzyme. Thus, a therapeutically effective amount of a compound of the invention is a quantity sufficient to modulate, regulate, or inhibit the activity of one or more PARP enzyme such that a disease condition that is mediated by that activity is reduced or alleviated. The effective amount of a given compound will vary depending upon factors such as the disease condition and its severity and the identity and condition (e.g., weight) of the mammal in need of treatment, but can nevertheless be routinely determined by one skilled in the art. “Treating” is intended to mean at least the mitigation of a disease condition in a mammal, including a human, that is affected, at least in part, by the activity of one or more PARP enzymes and includes: preventing the disease condition from occurring in a mammal, particularly when the mammal is found to be predisposed to having the disease condition but has not yet been diagnosed as having it; modulating and/or inhibiting the disease condition; and/or alleviating the disease condition. Exemplary disease condition includes cancer.

The activity of the compound of formula 1 as a modulator of PARP activity may be measured by any of the methods available to those skilled in the art, including in vivo and/or in vitro assays. Examples of suitable assays for activity measurements include those described in U.S. Pat. No. 6,495,541 and the specific examples of the present invention.

The present invention is directed to therapeutic methods of treating a disease condition mediated by PARP activity, for example, cancer and a variety of disease and toxic states that involve oxidative or nitric oxide induced stress and subsequent PARP hyperactivation. Such conditions include, but not limited to, neurologic and neurodegenerative disorders (eg, Parkinson's disease, Alzheimer's disease), cardiovascular disorders (eg, myocardial infarction, ischemia-reperfusion injury), diabetic vascular dysfunction, cisplatin-induced nephrotoxicity. The therapeutic methods of the present invention comprise administering to a mammal in need thereof a therapeutically effective amount of a pharmaceutical composition which comprises any of the polymorphic forms, or pharmaceutical compositions discussed above.

This invention also relates to a method for the treatment of abnormal cell growth in a mammal, including a human, comprising administering to said mammal an amount of the compound of formula 1, as defined above, or a pharmaceutically acceptable salt or solvate thereof, that is effective in treating abnormal cell growth.

In one embodiment of this method, the abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of the compound of formula 1, or a pharmaceutically acceptable salt or solvate thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of the compound of formula 1, as defined above, or a pharmaceutically acceptable salt or solvate thereof, that is effective in treating abnormal cell growth, and a pharmaceutically acceptable carrier. In one embodiment of said composition, said abnormal cell growth is cancer, including, but not limited to, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

The invention also relates to a pharmaceutical composition for the treatment of abnormal cell growth in a mammal, including a human, which comprises an amount of the compound of formula 1, as defined above, or a pharmaceutically acceptable salt or solvate thereof, that is effective in treating abnormal cell growth in combination with a pharmaceutically acceptable carrier and an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The invention also relates to a method for the treatment of a hyperproliferative disorder in a mammal which comprises administering to said mammal a therapeutically effective amount of the compound of formula 1, or a pharmaceutically acceptable salt or hydrate thereof, in combination with an anti-tumor agent selected from the group consisting antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, statins, and anti-androgens.

The present invention is also directed to combination therapeutic methods of treating a disease condition mediated by PARP activity, which comprises administering to a mammal in need thereof a therapeutically effective amount of a pharmaceutical composition which comprises any of the polymorphic forms, or pharmaceutical compositions discussed above, in combination with a therapeutically effective amount of one or more substances selected from anti-tumor agents, anti-angiogenesis agents, signal transduction inhibitors, and antiproliferative agents. Such substances include those disclosed in PCT Publication Nos. WO 00/38715, WO 00/38716, WO 00/38717, WO 00/38718, WO 00/38719, WO 00/38730, WO 00/38665, WO 00/37107 and WO 00/38786, the disclosures of which are incorporated herein by reference in their entireties.

Examples of anti-tumor agents include temozolomide (SCHERING), irinotecan (PFIZER), topotecan (GLAXO SMITHKLINE), cisplatin (BRISTOL MEYERS SQUIBB; AM PHARM PARTNERS; BEDFORD; GENSIA SICOR PHARMS; PHARMACHEMIE), and doxorubicin hydrochloride (AM PHARM PARTNERS; BEDFORD; GENSIA; SICOR PHARMS; PHARMACHEMIE; ADRIA; ALZA).

The combination therapeutic methods include administering the compound of formula 1 and an anti-tumor agent using any desire dosage regimen. For example, the regimens can be dependent on the combination agent as follows:

(a) the compound of formula 1, a pharmaceutically acceptable salt or solvate, or a mixture thereof, can be administered in an amount of from 1 to 48 mg/m² expressed as free base equivalent mass of the compound of formula 1, daily×5 days every 28 days 1 hour before 25-200 mg/m² temozolomide, preferably, 100-200 mg/m² temozolomide;

(b) the compound of formula 1, a pharmaceutically acceptable salt or solvate, or a mixture thereof, can be administered in an amount of from 1 to 48 mg/m² expressed as free base equivalent mass of the compound of formula 1, 1 hour before the irinotecan dose and 24 hours later.

Dose Ranges for Irinotecan:

62-125 mg/m² weekly ×4 weeks every 6 weeks

175-350 mg/m² every 3 weeks

90-180 mg/m² every 2 weeks.

(c) the compound of formula 1, a pharmaceutically acceptable salt or solvate, or a mixture thereof, can be administered in an amount of from 1 to 48 mg/m² expressed as free base equivalent mass of the compound of formula 1, daily ×5 days every 21 days, 1 hour before the topotecan dose.

Dose Range for Topotecan:

0.75-1.5 mg/m² daily ×5 days every 21 days

(d) the compound of formula 1, a pharmaceutically acceptable salt or solvate, or a mixture thereof, can be administered in an amount of from 1 to 48 mg/m² expressed as free base equivalent mass of the compound of formula 1, either once every 34 weeks or daily ×3-5 days every 34 weeks, 1 hour before the cisplatin dose.

Dose Ranges for Cisplatin:

10-100 mg/m² every 34 weeks

10-40 mg/m² daily ×3-5 days every 3-4 weeks.

(e) the compound of formula 1, a pharmaceutically acceptable salt or solvate, or a mixture thereof, can be administered in an amount of from 1 to 48 mg/m² expressed as free base equivalent mass of the compound of formula 1, 1 hour before the doxorubicin dose and 24 hours later.

Dose Range for Doxorubicin:

20-75 mg/m² every 21-28 days.

The combination therapeutic methods of the present invention may include administering the compound of formula 1 a pharmaceutically acceptable salt or solvate, or a mixture thereof, in an amount of from 1 to 48 mg/m² expressed as free base equivalent mass of the compound of formula 1, and an anti-tumor agent(s) using, for example, dosage regimens presented in Table 1.

TABLE 1

| Name | Regimen | Reference |
|---|---|---|
| Irinotecan | 125 mg/m² over 90 minutes, days 1, 8, 15, 22 | Saltz et al. N Engl J Med. |
| | Repeat every 6 weeks | 2000; 343: 905-914. |
| Irinotecan | 300 or 350 mg/m² IV over 90 minutes, day 1 | Cunningham et al. Lancet. |
| | Repeat every 3 weeks | 1998; 352: 1413-1418. |
| IFL | Irinotecan 125 mg/m² IV over 90 minutes, days 1, 8, 15, 22 | Saltz et al. N Engl J |
| Saltz regimen | LV 20 mg/m² IV bolus, days 1, 8, 15, 22 | Med.2000; 343: 905-914. |
| | 5-FU 500 mg/m² IV bolus, days 1, 8, 15, 22 | |
| | Repeat every 6 weeks | |
| Irinotecan + 5- | Irinotecan 180 mg/m² over 2 hours, day 1 | Douillard et al. Lancet. |
| FU/LV | LV 200 mg/m² IV over 2 hours prior to 5-FU, days 1 and 2 | 2000; 355: 1041-1047. |
| Douillard regimen | 5-FU 400 mg/m² IV bolus, then 600 mg/m² continuous infusion | |
| | over 22 hours, days 1 and 2 | |
| | Repeat every 2 weeks | |
| FOLFIRI | Irinotecan 180 mg/m² over 90 minutes, day 1 | André et al. Eur J |
| | LV 200 mg/m² over 2-hour infusion during irinotecan | Cancer.1999; 35: 1343-1347. |
| | 5-FU bolus 400 mg/m², then 2.4-3 g/m² continuous infusion | Touringand et al. J Clin |
| | over 46 hours, days 1 and 2 | Oncol. 2004; 23: 229-237. |
| | Repeat every 2 weeks | |
| Caplri | Capecitabine 1,000 mg/m² PO bid, days 1-14 | Grothey et al. Proc Am Soc |
| | Irinotecan 100 mg/m², days 1 and 8 | Clin Oncol. 2003; 22: 255. |
| | Repeat every 22 days | Abstract 1022. |
| XELIRI | Irinotecan 250 mg/m² IV, day 1 | Patt et al. Proc Am Soc Clin |
| | Capecitabine 1,000 mg/m² PO bid, evening day 1-morning day | Oncol. 2004; 23; 271. |
| | 15 | Abstract 3602. |
| | Repeat every 3 weeks | |
| IROX | Irinotecan 200 mg/m² IV over 90 minutes, day 1 | Goldberg et al. J Clin Oncol. |
| | Oxaliplatin 85 mg/m² IV over 2 hours, day 1 | 2004; 22: 23-30. |
| | Repeat every 3 weeks | |
| IFL + Bevacizumab | Irinotecan 125 mg/m² IV over 90 minutes, days 1, 8, 15, 22 | Hurwiz et al. N Engl J Med. |
| | LV 20 mg/m² IV, days 1, 8, 15, 22 | 2004; 350: 2335-2342. |
| | 5-FU 500 mg/m² IV, days 1, 8, 15, 22 | |
| | Repeat every 6 weeks | |
| | Bevacizumab 5 mg/kg IV over 90 minutes* following | |
| | chemotherapy, day 1 | |
| | Repeat every 2 weeks | |

CRC = colorectal cancer;
5-FU = 5-fluorouracil;
LV = leucovorin.
*If first infusion is well tolerated, subsequent infusions may be administered over 60 minutes and then 30 minutes.

The dosing schemes listed in Table 1 can be modified. For example, irinotecan may be given at a dose of 50-350 mg/m²; 5-FU may be given at a dose of 370 mg/m²-3.0 g. LV may be given at 20-500 mg/m².

The combination therapeutic methods of the present invention which include administering the compound of formula 1, a pharmaceutically acceptable salt or solvate, or a mixture thereof, in an amount of from 1 to 48 mg/m² expressed as free base equivalent mass of the compound of formula 1, and an anti-tumor agent(s), may be used, for example, in treatment patients who, for example, failed treatment with the regimens presented in Table 2.

TABLE 2

| Name | Regimen | Reference |
|---|---|---|
| FOLFOX4 | Oxaliplatin 85 mg/m² IV over 2 hours, day 1 | de Gramont et al. J Clin |
| | LV 200 mg/m² IV over 2 hours, days 1 and 2 | Oncol.2000; 18: 2938-2947. |
| | 5-FU 400 mg/m² IV bolus, then 600 mg/m² IV over 22 hours, | Rothenberg et al. J Clin |
| | days 1 and 2 | Oncol. 2003; 21: 2059-2069. |
| | Repeat every 2 weeks | |
| FOLFOX6 | Oxaliplatin 100 mg/m² IV over 2 hours, day 1 | Maindrault-Goebel et al. Eur |
| | LV 200 mg/m² IV over 2 hours, day 1 | J Cancer. 1999; 35: 1338-1342. |
| | 5-FU 400 mg/m² IV bolus, then 2.4-3 g/m² over 46 hours, | Tournigand et al. J Clin |
| | continuous infusion | Oncol. 2004; 23: 229-237. |
| | Repeat every 2 weeks | |
| mFOLFOX6 | Oxaliplatin 85 mg/m² IV over 2 hours, day 1 | Cheeseman et al. Br J |
| | LV 175 mg/m² IV over 2 hours, day 1 | Cancer. 2002; 87: 393-399. |
| | 5-FU 400 mg/m² IV bolus, then 2.4-3 g/m² over 46 hours, | |
| | continuous infusion | |
| | Repeat every 2 weeks | |
| FOLFOX7 | Oxaliplatin 130 mg/m² IV over 2 hours, day 1 | André et al. Proc Am Soc |
| | LV 400 mg/m² IV over 2 hours | Clin Oncol. 2003; 22: 253. |
| | 5-FU 2,400 mg/m² IV over 46 hours, continuous infusion | Abstract 1016. |
| | Repeat every 2 weeks for 6 cycles | |
| FLOX | Oxaliplatin 85 mg/m² IV over 2 hours, days 1, 15, 29 | Smith et al. Proc Am Soc |
| | LV 500 mg/m² IV over 2 hours, days 1, 8, 15, 22, 29, 36 | Clin Oncol. 2003; 22: 294. |
| | 5-FU 500 mg/m² IV bolus 1 hour after start of LV, days 1, 8, 15, | Abstract 1181. |
| | 22, 29, 36 | |
| | Repeat every 8 weeks for 3 cycles | |
| FUFOX | Oxaliplatin 60 mg/m² IV over 2 hours, days 1, 8, 15, 22 | Moehler et al. Z |
| | LV 500 mg/m² IV over 2 hours, days 1, 8, 15, 22 | Gastroenterol. 2002; 40: 957-964. |
| | 5-FU 2.6 g/m² IV over 24 hours, continuous infusion, | |
| | days 1, 8, 15, 22 | |
| | Repeat every 36 days | |
| bFOL | Oxaliplatin 85 mg/m² IV over 2 hours, every 2 weeks | Hochester et al. J Clin |
| | LV 20 mg/m² IV over 10-20 minutes, days 1, 8, 15 | Oncol. 2003; 21: 2703-2707. |
| | 5-FU 500 mg/m² IV bolus, days 1, 8, 15 | |
| | Repeat every 28 days | |
| FOLFOX 4 + Bevacizumab | Oxaliplatin 85 mg/m² IV over 2 hours, day 1 | Benson et al. Proc Am Soc |
| | LV 200 mg/m² IV over 2 hours, days 1 and 2 | Clin Oncol. 2003; 22: 243. |
| | 5-FU 400 mg/m² IV bolus, then 600 mg/m² IV over 22 hours, | Abstract 975. |
| | days 1 and 2 | |
| | Bevacizumab 10 mg/kg IV over 90 minutes, *day 1 | |
| | Repeat every 2 weeks | |
| FOLFOX4 + Cetuximab | Oxaliplatin 85 mg/m² IV over 2 hours, day 1 | Tabernero et al. Proc Am |
| | LV 200 mg/m² IV over 2 hours, days 1 and 2 | Soc Clin Oncol. |
| | 5-FU 400 mg/m² IV bolus, then 600 mg/m² IV over 22 hours, | 2004; 23: 248. Abstract 3512. |
| | days 1 and 2 | |
| | Repeat every 2 weeks | |
| | Cetuximab 400 mg/m² IV over 2 hours week 1 followed by | |
| | 250 mg/m² IV over 60 minutes weekly | |

The dosage units are represented in mg per m² of BSA. For example, the Mosteller formula, the DuBois and DuBois formula, the Haycock formula, the Gehan and George formula, the Boyd formula are applicable for measuring BSA (Mosteller RD: Simplified Calculation of Body Surface Area. N Engl J Med October 1987 22;317(17):1098; DuBois D; DuBois E F: A formula to estimate the approximate surface area if height and weight be known. Arch Int Med 1916 17:863-71; Haycock G. B., Schwartz G. J.,Wisotsky D. H. Geometric method for measuring body surface area: A height weight formula validated in infants, children and adults. The Journal of Pediatrics 1978 93:1:62-66; Gehan E A, George S L, Estimation of human body surface area from height and weight. Cancer Chemother Rep 1970 54:225-35; Boyd E, The growth of the surface area of the human body. Minneapolis: university of Minnesota Press, 1935; Lam T K, Leung D T: More on simplified calculation of body-surface area. N Engl J Med April 1988 28;318(17): 1130).

Additional examples of anti-tumor agents include antiproliferative agents, kinase inhibitors, angiogenesis inhibitors, growth factor inhibitors, cox-I inhibitors, cox-II inhibitors, mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, statins, and anti-androgens.

In one embodiment of the present invention the anti-tumor agent used in conjunction with the compound of formula 1 and pharmaceutical compositions described herein is an anti-angiogenesis agent, kinase inhibitor, pan kinase inhibitor or growth factor inhibitor.

Preferred pan kinase inhibitors include SU-11248, described in U.S. Pat. No. 6,573,293 (Pfizer, Inc, NY, USA).

Anti-angiogenesis agents, include but are not limited to the following agents, such as EGF inhibitor, EGFR inhibitors, VEGF inhibitors, VEGFR inhibitors, TIE2 inhibitors, IGF1R inhibitors, COX-II (cyclooxygenase II) inhibitors, MMP-2 (matrix-metalloprotienase 2) inhibitors, and MMP-9 (matrix-metalloprotienase 9) inhibitors.

Preferred VEGF inhibitors, include for example, Avastin (bevacizumab), an anti-VEGF monoclonal antibody of Genentech, Inc. of South San Francisco, Calif.

Additional VEGF inhibitors include CP-547,632 (Pfizer Inc., NY, USA), AG13736 (Pfizer Inc.), ZD-6474 (AstraZeneca), AEE788 (Novartis), AZD-2171), VEGF Trap (Regeneron,/Aventis), Vatalanib (also known as PTK-787, ZK-222584: Novartis & Schering AG), Macugen (pegaptanib octasodium, NX-1838, EYE-001, Pfizer Inc./Gilead/Eyetech), IM862 (Cytran Inc. of Kirkland, Wash., USA); and angiozyme, a synthetic ribozyme from Ribozyme (Boulder, Colo.) and Chiron (Emeryville, Calif.) and combinations thereof. VEGF inhibitors useful in the practice of the present invention are disclosed in U.S. Pat. Nos. 6,534,524 and 6,235,764, both of which are incorporated in their entirety for all purposed. Particularly preferred VEGF inhibitors include CP-547,632, AG13736, Vatalanib, Macugen and combinations thereof.

Additional VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 6,534,524 (discloses AG13736), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. patent applications: Ser. No. 09/221946 (filed Dec. 28, 1998); Ser. No. 09/454058 (filed Dec. 2, 1999); 09/501163 (filed Feb. 9, 2000); Ser. No. 09/539930 (filed Mar. 31, 2000); Ser. No. 09/202796 (filed May 22, 1997); Ser. No. 09/384339 (filed Aug. 26, 1999); and Ser. No. 09/383755 (filed Aug. 26, 1999); and the compounds disclosed and claimed in the following U.S. provisional patent applications: 60/168207 (filed Nov. 30, 1999); 60/170119 (filed Dec. 10, 1999); 60/177718 (filed Jan. 21, 2000); 60/168217 (filed Nov. 30, 1999), and 60/200834 (filed May 1, 2000). Each of the foregoing patent applications and provisional patent applications is herein incorporated by reference in their entirety.

PDGRr inhibitors include but not limited to those disclosed international patent application publication number WO01/40217, published Jul. 7, 2001 and international patent application publication number WO2004/020431, published Mar. 11, 2004, the contents of which are incorporated in their entirety for all purposes.

Preferred PDGFr inhibitors include Pfizer's CP-673,451 and CP-868,596 and its pharmaceutically acceptable salts.

Preferred GARF inhibitors include Pfizer's AG-2037 (pelitrexol and its pharmaceutically acceptable salts. GARF inhibitors useful in the practice of the present invention are disclosed in U.S. Pat. No. 5,608,082 which is incorporated in its entirety for all purposed.

Examples of useful COX-11 inhibitors which can be used in conjunction with the compound of formula land pharmaceutical compositions described herein include CELEBREX™ (celecoxib), parecoxib, deracoxib, ABT-963, MK-663 (etoricoxib), COX-189 (Lumiracoxib), BMS 347070, RS 57067, NS-398, Bextra (valdecoxib), paracoxib, Vioxx (rofecoxib), SD-8381, 4-Methyl-2-(3,4-dimethylphenyl)-1-(4-sulfamoyl-phenyl)-1H-pyrrole, 2-(4-Ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additonally, COX-II inhibitors are disclosed in U.S. patent application Ser. Nos. 10/801,446 and 10/801,429, the contents of which are incorporated in their entirety for all purposes.

In one preferred embodiment the anti-tumor agent is celecoxib as disclosed in U.S. Pat. No. 5,466,823, the contents of which are incorporated by reference in its entirety for all purposes. The structure for Celecoxib is shown below:

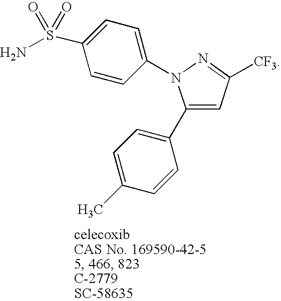

In one preferred embodiment the anti-tumor agent is valecoxib as disclosed in U.S. Pat. No. 5,633,272, the contents of which are incorporated by reference in its entirety for all purposes. The structure for valdecoxib is shown below:

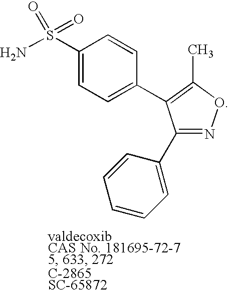

In one preferred embodiment the anti-tumor agent is parecoxib as disclosed in U.S. Pat. No. 5,932,598, the contents of which are incorporated by reference in its entirety for all purposes. The structure for paracoxib is shown below:

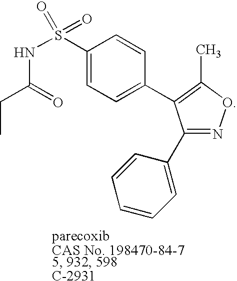

In one preferred embodiment the anti-tumor agent is deracoxib as disclosed in U.S. Pat. No. 5,521,207, the contents of which are incorporated by reference in its entirety for all purposes. The structure for deracoxib is shown below:

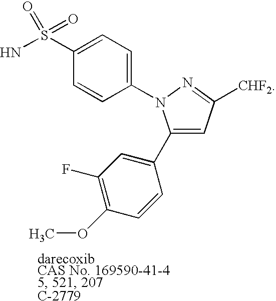

In one preferred embodiment the anti-tumor agent is SD-8381 as disclosed in U.S. Pat. No. 6,034,256, the contents of which are incorporated by reference in its entirety for all purposes. The structure for SD-8381 is shown below:

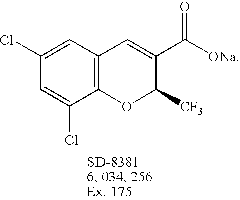

In one preferred embodiment the anti-tumor agent is ABT-963 as disclosed in International Publication Number WO 2002/24719, the contents of which are incorporated by reference in its entirety for all purposes. The structure for ABT-963 is shown below:

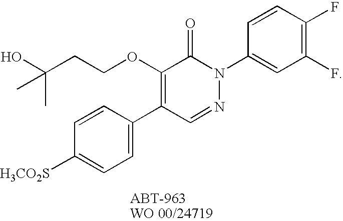

In one preferred embodiment the anti-tumor agent is rofecoxib as shown below:

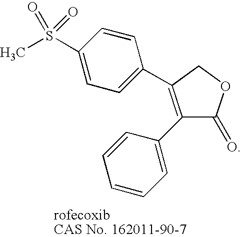

In one preferred embodiment the anti-tumor agent is MK-663 (etoricoxib) as disclosed in International Publication Number WO 1998/03484, the contents of which are incorporated by reference in its entirety for all purposes. The structure for etoricoxib is shown below:

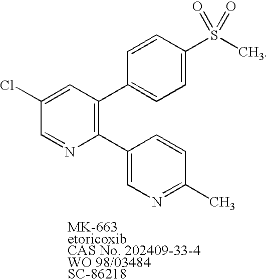

In one preferred embodiment the anti-tumor agent is COX-189 (Lumiracoxib) as disclosed in International Publication Number WO 1999/11605, the contents of which are incorporated by reference in its entirety for all purposes. The structure for Lumiracoxib is shown below:

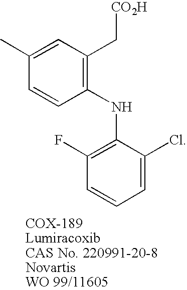

In one preferred embodiment the anti-tumor agent is BMS-347070 as disclosed in U.S. Pat. No. 6,180,651, the contents of which are incorporated by reference in its entirety for all purposes. The structure for BMS-347070 is shown below:

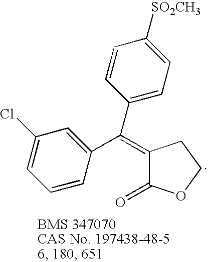

In one preferred embodiment the anti-tumor agent is NS-398 (CAS 123653-11-2). The structure for NS-398 (CAS 123653-11-2) is shown below:

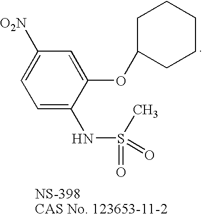

In one preferred embodiment the anti-tumor agent is RS 57067 (CAS 17932-91-3). The structure for RS-57067 (CAS 17932-91-3) is shown below:

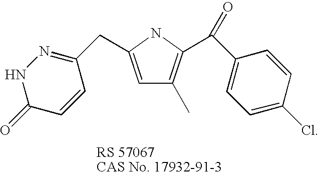

In one preferred embodiment the anti-tumor agent is 4-Methyl-2-(3,4-dimethylphenyl)-1-(4-sulfamoyl-phenyl)-1H-pyrrole. The structure for 4-Methyl-2-(3,4-dimethylphenyl)-1-(4-sulfamoyl-phenyl)-1H-pyrrole is shown below:

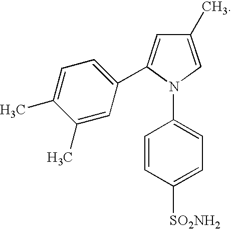

In one preferred embodiment the anti-tumor agent is 2-(4-Ethoxyphenyl)-4-methyl-1-(4-sulfamoylphenyl)-1H-pyrrole. The structure for 2-(4-Ethoxyphenyl)-4-methyl-1-(4-sulfamoylphenyl)-1H-pyrrole is shown below:

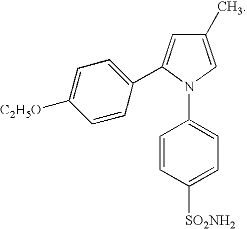

In one preferred embodiment the anti-tumor agent is meloxicam. The structure for meloxicam is shown below:

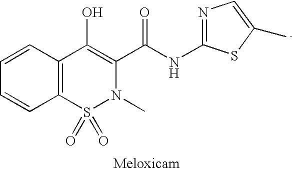

Other useful inhibitors as anti-tumor agents used in conjunction with the compound of formula land pharmaceutical compositions described herein include aspirin, and non-steroidal anti-inflammatory drugs (NSAIDs) which inhibit the enzyme that makes prostaglandins (cyclooxygenase I and II), resulting in lower levels of prostaglandins, include but are not limited to the following, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred COX-I inhibitors include ibuprofen (Motrin), nuprin, naproxen (Aleve), indomethacin (Indocin), nabumetone (Relafen) and combinations thereof.

Targeted agents used in conjunction with the compound of formula land pharmaceutical compositions described herein include EGFr inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), EMD-7200 (Merck AG), ABX-EGF (Amgen Inc. and Abgenix Inc.), HR3 (Cuban Government), IgA antibodies (University of Erlangen-Nuremberg), TP-38 (IVAX), EGFR fusion protein, EGF-vaccine, anti-EGFr immunoliposomes (Hermes Biosciences Inc.) and combinations thereof

Preferred EGFr inhibitors include Iressa, Erbitux, Tarceva and combinations thereof.

The present invention also relates to anti-tumor agents selected from pan erb receptor inhibitors or ErbB2 receptor inhibitors, such as CP-724,714 (Pfizer, Inc.), CI-1033 (canertinib, Pfizer, Inc.), Herceptin (trastuzumab, Genentech Inc.), Omitarg (2C4, pertuzumab, Genentech Inc.), TAK-165 (Takeda), GW-572016 (Ionafarnib, GlaxoSmithKline), GW-282974 (GlaxoSmithKline), EKB-569 (Wyeth), PKI-166 (Novartis), dHER2 (HER2 Vaccine, Corixa and GlaxoSmithKline), APC8024 (HER2 Vaccine, Dendreon), anti-HER2/neu bispecific antibody (Decof Cancer Center), B7.her2.IgG3 (Agensys), AS HER2 (Research Institute for Rad Biology & Medicine), trifuntional bispecific antibodies (University of Munich) and mAB AR-209 (Aronex Pharmaceuticals Inc) and mAB 2B-1 (Chiron) and combinations thereof. Preferred erb selective anti-tumor agents include Herceptin, TAK-165, CP-724,714, ABX-EGF, HER3 and combinations thereof.

Preferred pan erbb receptor inhibitors include GW572016, CI-1033, EKB-569, and Omitarg and combinations thereof.

Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277 each of which are herein incorporated by reference in their entirety.

Additionally, other anti-tumor agents may be selected from the following agents, BAY-43-9006 (Onyx Pharmaceuticals Inc.), Genasense (augmerosen, Genta), Panitumumab (Abgenix/Amgen), Zevalin (Schering), Bexxar (Corixa/GlaxoSmithKline), Abarelix, Alimta, EPO 906 (Novartis), discodermolide (XAA-296), ABT-510 (Abbott), Neovastat (Aetema), enzastaurin (Eli Lilly), Combrestatin A4P (Oxigene), ZD-6126 (AstraZeneca), flavopiridol (Aventis), CYC-202 (Cyclacel), AVE-8062 (Aventis), DMXAA (Roche/Antisoma), Thymitaq (Eximias), Temodar (temozolomide, Schering Plough) and Revilimd (Celegene) and combinations thereof.

Other anti-tumor agents may be selected from the following agents, CyPat (cyproterone acetate), Histerelin (histrelin acetate), Plenaixis (abarelix depot), Atrasentan (ABT-627), Satraplatin (JM-216), thalomid (Thalidomide), Theratope, Temilifene (DPPE), ABI-007 (paclitaxel), Evista (raloxifene), Atamestane (Biomed-777), Xyotax (polyglutamate paclitaxel), Targetin (bexarotine) and combinations thereof. Additionally, other anti-tumor agents may be selected from the following agents, Trizaone (tirapazamine), Aposyn (exisulind), Nevastat (AE-941), Ceplene (histamine dihydrochloride), Orathecin (rubitecan), Virulizin, Gastrimmune (G17DT), DX-8951f (exatecan mesylate), Onconase (ranpirnase), BEC2 (mitumoab), Xcytrin (motexafin gadolinium) and combinations thereof.

Further anti-tumor agents may selected from the following agents, CeaVac (CEA), NeuTrexin (trimetresate glucuronate) and combinations thereof.

Additional anti-tumor agents may selected from the following agents, OvaRex (oregovomab), Osidem (IDM-1), and combinations thereof.

Additional anti-tumor agents may selected from the following agents, Advexin (ING 201), Tirazone (tirapazamine), and combinations thereof.

Additional anti-tumor agents may selected from the following agents, RSR13 (efaproxiral), Cotara (131I chTNT 1/b), NBI-3001 (IL-4) and combinations thereof. Additional anti-tumor agents may selected from the following agents, Canvaxin, GMK vaccine, PEG Interon A, Taxoprexin (DHA/paciltaxel) and combinations thereof.

Other preferred anti-tumor agents include Pfizer's MEK1/2 inhibitor PD325901, Array Biopharm's MEK inhibitor ARRY-142886, Bristol Myers' CDK2 inhibitor BMS-387,032, Pfizer's CDK inhibitor PD0332991 and AstraZeneca's AXD-5438 and combinations thereof.

Additionally, mTOR inhibitors may also be utilized such as CCI-779 (Wyeth) and rapamycin derivatives RAD001 (Novartis) and AP-23573 (Ariad), HDAC inhibitors SAHA (Merck Inc./Aton Pharmaceuticals) and combinations thereof.

Additional anti-tumor agents include aurora 2 inhibitor VX-680 (Vertex), Chk1/2 inhibitor XL844 (Exilixis).

The following cytotoxic agents, , e.g., one or more selected from the group consisting of epirubicin (Ellence), docetaxel (Taxotere), paditaxel, Zinecard (dexrazoxane), rituximab (Rituxan) imatinib mesylate (Gleevec), and combinations thereof, may be used in conjunction with the compound of formula land pharmaceutical compositions described herein.

The invention also contemplates the use of the compounds of the present invention together with hormonal therapy, including but not limited to, exemestane (Aromasin, Pfizer Inc.), leuprorelin (Lupron or Leuplin, TAP/Abbott/Takeda), anastrozole (Arimidex, Astrazeneca), gosrelin (Zoladex, AstraZeneca), doxercalciferol, fadrozole, formestane, tamoxifen citrate (tamoxifen, Nolvadex, AstraZeneca), Casodex (AstraZeneca), Abarelix (Praecis), Trelstar, and combinations thereof.

The invention also relates to hormonal therapy agents such as anti-estrogens including, but not limited to fulvestrant, toremifene, raloxifene, lasofoxifene, letrozole (Femara, Novartis), anti-androgens such as bicalutamide, flutamide, mifepristone, nilutamide, Casodex®)(4′-cyano-3-(4-fluorophenylsulphonyl)-2-hydroxy-2-methyl-3′-(trifluoromethyl)propionanilide, bicalutamide) and combinations thereof.

Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

Particularly preferred cytotoxic agents include Camptosar, Erbitux, Iressa, Gleevec, Taxotere and combinations thereof.

The following topoisomerase I inhibitors may be utilized as anti-tumor agents camptothecin, irinotecan HCl (Camptosar), edotecarin, orathecin (Supergen), exatecan (Daiichi), BN-80915 (Roche) and combinations thereof.

Particularly preferred toposimerase II inhibitors include epirubicin (Ellence).

The compounds of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers.

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, Paraplatin (carboplatin), eptaplatin, lobaplatin, nedaplatin, Eloxatin (oxaliplatin, Sanofi) or satrplatin and combinations thereof. Particularly preferred alkylating agents include Eloxatin (oxaliplatin).

Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, Alimta (premetrexed disodium, LY231514, MTA), Gemzar (gemcitabine, Eli Lilly), fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

Antibiotics include intercalating antibiotics but are not limited to: aclarubicin, actinomycin D, amrubicin, annamycin, adriamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin, zinostatin and combinations thereof.

Plant derived anti-tumor substances include for example those selected from mitotic inhibitors, for example vinblastine, docetaxel (Taxotere), paclitaxel and combinations thereof.

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, topotecan, and combinations thereof.

Preferred cytotoxic topoisomerase inhibiting agents include one or more agents selected from the group consisting of camptothecin, 10-hydroxycamptothecin, 9-aminocamptothecin, irinotecan HCl (Camptosar), edotecarin, epirubicin (Ellence), etoposide, SN-38, topotecan, and combinations thereof.

Immunologicals include interferons and numerous other immune enhancing agents. Interferons include interferon alpha, interferon alpha-2a, interferon, alpha-2b, interferon beta, interferon gamma-1a, interferon gamma-1b (Actimmune), or interferon gamma-n1 and combinations thereof. Other agents include filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab (Y-muHMFG1), Provenge (Dendreon) and combinations thereof.

Biological response modifiers are agents that modify defense mechanisms of living organisms or biological responses, such as survival, growth, or differentiation of tissue cells to direct them to have anti-tumor activity. Such agents include krestin, lentinan, sizofiran, picibanil, ubenimex and combinations thereof.

Other anticancer agents include alitretinoin, ampligen, atrasentan bexarotene, bortezomib. Bosentan, calcitriol, exisulind, finasteride,fotemustine, ibandronic acid, miltefosine, mitoxantrone, I-asparaginase, procarbazine, dacarbazine, hydroxycarbamide, pegaspargase, pentostatin, tazarotne, Telcyta (TLK-286, Telik Inc.), Velcade (bortemazib, Millenium), tretinoin, and combinations thereof.

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain, Vitaxin and combinations thereof.

Platinum-coordinated compounds include but are not limited to, cisplatin, carboplatin, nedaplatin, oxaliplatin, and combinations thereof.

Camptothecin derivatives include but are not limited to camptothecin, 10-hydroxycamptothecin, 9-aminocamptothecin, irinotecan, SN-38, edotecarin, topotecan and combinations thereof.

Other antitumor agents include mitoxantrone, I-asparaginase, procarbazine, dacarbazine, hydroxycarbamide, pentostatin, tretinoin and combinations thereof.

Anti-tumor agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736; and anti-proliferative agents such as other farnesyl protein transferase inhibitors, for example the farnesyl protein transferase inhibitors. Additional, specific CTLA4 antibodies that can be used in the present invention include those described in U.S. Provisional Application 60/113,647 (filed Dec. 23, 1998), U.S. Pat. No. 6,682,736 both of which are herein incorporated by reference in their entirety.

Specific IGF1R antibodies that can be used in the present invention include those described in International Patent Application No. WO 2002/053596, which is herein incorporated by reference in its entirety.

Specific CD40 antibodies that can be used in the present invention include those described in International Patent Application No. WO 2003/040170 which is herein incorporated by reference in its entirety.

Gene therapy agents may also be employed as anti-tumor agents such as TNFerade (GeneVec), which express TNFalpha in response to radiotherapy.

In one embodiment of the present invention statins may be used in conjunction with the compound of formula land pharmaceutical compositions. Statins (HMG-COA reducatase inhibitors) may be selected from the group consisting of Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof.

In a preferred embodiment the statin is selected from the group consisting of Atovorstatin and Lovastatin, derivatives and combinations thereof.

Other agents useful as anti-tumor agents include Caduet.

The methods include administering the compound of formula 1 using any desire dosage regimen. In one specific embodiment, the compound is administered once per day, although more or less frequent administration is within the scope of the invention. The compound of formula 1 can be administered on the same schedule as the cytotoxic with which it is being co-administered. In cases where the half-life of the cytotoxic agent is long (ie, >10 hours) consideration can be given to administering the compound of formula 1 alone on the day after the cytotoxic is administered as well. The compound of formula 1 can be administered to the mammal, including a human, preferably by intravenous injection over a period of 30 minutes.

In another embodiment of the present invention the compound of formula 1 is used as a radiosensitizer that enhances the efficacy of radiotherapy. In according to the present invention, the compound of formula 1 can be used in combination with any kind of radiotherapy including external beam radiotherapy (XBRT) or teletherapy, brachytherapy or sealed source radiotherapy, unsealed source radiotherapy and radio-immunotherapy. In according to the present invention, to maximize clinical tumor response, radiation is usually given as daily fractions of 2-4 Gy to a total dose of 50-60 Gy. One with the ordinary skills in the art will appreciate that precise protocols will differ dependent on the disease site and whether radiation is administered with curative intent or as palliative treatment. Further information regarding different kinds of radiotherapy can be found, for example, in “Absorbed Dose Determination in External Beam Radiotherapy,” International Atomic Energy Agency, Vienna, 2000, Technical Reports Series No. 398; “Principles and Practice of Brachytherapy: Using Afterloading Systems,” Joslin et al. (Eds.), Arnold Publishers, 1st Edition, 2001; “Proton Therapy and Radiosurgery,” Smit et al. (Eds.), Springer-Verlag Telos, 1st Edition, 2000; Greig et al. “Treatment with unsealed radioisotopes,” Br. Med. Bull., 1973, 29(1):63-68; “Radioimmunotherapy of Cancer,” Abrams et al. (Eds.), Marcel Dekker, 1st Edition, 2000. U.S. Pat. No. 6,649,645 teaches combination therapy of radiation and cyclooxygenase-2 inhibitor for treatment of neoplasia disorders.

In another embodiment of the present invention the compound of formula 1 is used in combination with radiotherapy and at least one anti-tumor agent.

In another embodiment of the present invention the compound of formula 1 is used in combination with radiotherapy and at least one radiopotentiator such as, for example, growth factor receptor antagonists.

The methods and compositions of the present invention provide one or more benefits. A combination of the compound of formula 1 with chemotherapy or radiation therapy of the present invention may be administered at a low dose, that is, at a dose lower than has been conventionally used in clinical situations for each of the individual components administered alone. A benefit of lowering the dose of the chemotherapies or radiation therapies of the present invention administered to a mammal includes a decrease in the incidence of adverse effects associated with higher dosages. By lowering the incidence of adverse effects, an improvement in the quality of life of a patient undergoing treatment for cancer is contemplated. Further benefits of lowering the incidence of adverse effects include an improvement in patient compliance, and a reduction in the number of hospitalizations needed for the treatment of adverse effects.

Alternatively, the methods and combination of the present invention can also maximize the therapeutic effect at higher doses.

### EXAMPLES

The examples and preparations provided below further illustrate and exemplify the combinations, dosage forms and methods of the present invention. It is to be understood that the scope of the present invention is not limited in any way by the scope of the following examples.

Materials

All chemicals were obtained from Sigma (Poole, Dorset, UK) unless stated otherwise. Dulbecco's phosphate buffer saline (PBS) was obtained from Gibco (Paisley, UK), sucrose, sodium hydroxide and potassium chloride were supplied by BDH (Lufterworth, UK) and digitonin by Boehringer Mannheim (Roche Diagnostics, Lewes, UK). The BCA protein assay kit (Pierce, Perbio Science, Rockford, Ill., USA) was used for protein concentration determinations. Milk powder was obtained from Marvel Premier Brands UK Ltd (Spalding, UK), and ECL Western Blot Detection kits from Amersham (Little Chalfont, UK). Nycomed® Lymphoprep was obtained from Axis-Shield (Oslo, Norway) and EDTA blood collection tubes from BD Vacutainer (Plymouth, UK). The 10H mouse monoclonal primary antibody was generously supplied by Professor Alexander Bürkle, and the goat anti-mouse secondary antibody (HRP-conjugated) was obtained from DAKO (Ely, UK). The oligonucleotide used to stimulate PARP activity was initially synthesised by Dr J Lunec (Northern Institute for Cancer Research, Newcastle), and subsequent supplies were obtained from Invitrogen (Glasgow, UK). Purified poly(ADP ribose) (PAR) polymer was obtained from BIOMOL Research Lab (Plymouth, Pa., USA).

Tissue Culture of SW620 and L1210 (Quality Control) Cells

Cells were maintained in RPMI 1640 medium (Sigma) supplemented with 10% (v/v) foetal calf serum (Invitrogen) and 1 U/ml penicillin-streptomycin solution (Sigma), in a Hereus incubator (Fischer Scientific, Manchester, UK) maintained at 37° C. in a humidified atmosphere of 5% CO₂ in air. L1210 cells used were obtained from ATCC (American Type Culture Collection, Manassas, Va.) and grown as a suspension to a density of approximately 6×10⁵/ml at harvesting, to ensure exponential growth. Aliquots of 1×10⁶ cells for use as quality control samples were resuspended in 1 ml of medium plus 10% (v/v) DMSO and 10% (v/v) foetal calf serum and frozen at −80° C.

Preparation of Tumour Xeno-Graft Samples

Tumours were excised and were snap frozen in liquid nitrogen and stored at −80° C. until homogenised for analysis. The specimen was defrosted on ice and the wet weight documented. The tissue was homogenised using a Pro 2000 instrument (Pro Scientific Inc, Monroe, Conn., USA) in 3 volumes (i.e. 1 mg plus 3 μl isotonic buffer −7 mM Hepes, 26 mM KCl, 0.1 mM dextran, 0.4 mM EGTA, 0.5 mM MgCl₂, 45 mM sucrose, pH 7.8), giving a homogenate with an overall dilution of 1 in 4. The homogenate was kept on ice throughout the process, and homogenisation was performed in 10 second bursts to prevent undue warming of the sample. Prior to assay the samples were further diluted with isotonic buffer where necessary to give a final dilution of 1 in 40 for [³²P]NAD incorporation assay or 1 in 1000 for immunoblot aaasy.

Preparation of PBL and Tumour Samples.

Whole blood was collected into EDTA vacutainers and human PBLs were obtained by lymphopreparation according to the manufacturers instructions. Tumour biopsies were collected from the operating theatre in a sterile container and placed immediately on ice. Within 30 minutes tumour samples were snap frozen in liquid nitrogen and stored at −80° C. until homogenised for analysis. The specimen was defrosted on ice and the wet weight documented. For weights over 100 mg the tissue was homogenised using a Pro 2000 instrument (Pro Scientific Inc, Monroe, Conn., USA) in 3 volumes (i.e. 1 mg plus 3 μl isotonic buffer −7 mM Hepes, 26 mM KCl, 0.1 mM dextran, 0.4 mM EGTA, 0.5 mM MgCl₂, 45 mM sucrose, pH 7.8), giving a homogenate with an overall dilution of 1 in 4. Where smaller samples had been obtained they were homogenised in 99 or 999 volumes, giving final dilutions of 1 in 100 and 1 in 1000, respectively. The homogenate was kept on ice throughout the process, and homogenisation was performed in 10 second bursts to prevent undue warming of the sample. Unless assayed on the day of homogenisation, samples were re-frozen to −80° C. and stored at this temperature until analysed. Prior to assay the samples were further diluted with isotonic buffer where necessary to give a final dilution of 1 in 1000.

PARP Assay Using [³²P]NAD Incorporation

As described in: Calabrese C R, Almassy R, Barton S, Batey M A, Calvert A H, Canan-Koch S, Durkacz B W, Hostomsky Z, Kumpf R A, Kyle S, Li J, Maegley K, Newell D R, North M, Notarianni E, Strafford I J, Skalitzky D, Thomas H D , Wang L-Z, Webber S E, Williams K J and Curtin N J. Preclinical evaluation of a novel poly(ADP-ribose) polymerase-1 (PARP-1) inhibitor, AG14361, with significant anticancer chemo- and radio-sensitization activity. JNCI 96 56-67 (2004) and Bowman K J, Newell D R, Calvert A H and Curtin N J. Differential effects of the poly(ADP-ribose) polymerase (PARP) inhibitor NU1025 on topoisomerase I and II inhibitor cytotoxicity. Br. J Cancer 84 106-112 (2001). based on previously published methods (Halldorsson H., Gray D. A., and Shall S. (1978). Poly(ADP-ribose)polymerase activity in nucleotide permeable cells. FEBS Letters 85: 349-352, Grube, K., Kupper, J. H. & Bürkle, A. Direct stimulation of poly(ADP-ribose) polymerase in permeabilised cells by double-stranded DNA oligomers. Anal. Biochemistry. 1991; 193: 236-239)

PARP inhibition was determined in digitonin (0.15 mg/ml)-permeabilised cells (8×10⁵-^{1×10}⁶/reaction), stimulated with exogenously added 12-mer blunt-ended DNA double stranded oligonucleotide (2.5 μg/ml), by measuring inhibition of 75 μM NAD⁺+[³²P]NAD⁺ (Amersham), incorporation into cellular macromolecules during a 6 min incubation at 25° C. then precipitated by ice-cold 10% TCA, 10% NaPPi (w/v) as described previously. Briefly, cells were suspended in hypotonic buffer (9 mM HEPES pH 7.8, 4.5% (v/v) dextran, 4.5 mM MgCl₂ and 5 mM DTT) at 1.5×10⁷/ml on ice for 30 minutes then 9 vol of isotonic buffer (40 mM HEPES pH 7.8, 130 mM KCl, 4% (v/v) dextran, 2 mM EGTA, 2.3 mM MgCl₂, 225 mM sucrose and 2.5 mM DTT) was added. The reaction was started by adding 300 μl cells to 100 μl 300 μM NAD⁺ containing [³²P]-NAD⁺ (Amersham, UK), and terminated by the addition of 2 ml ice cold 10% (w/v) TCA+10% (w/v) sodium pyrophosphate. After 30 min on ice the precipitated ³²P—labelled ADP-ribose polymers were filtered on Whatman GC/C filters (Whatman Intemational Ltd, Kent, UK), washed 5 times with 1% (v/v) TCA/1% (v/v) sodium pyrophosphate, dried and counted. PARP inhibitory IC₅₀ values were calculated from computer-fitted curves (GraphPad Software, Inc., San Diego, Calif.).

Tumour homogenates were assayed in a similar manner; however, the homogenisation process introduces sufficient DNA damage to maximally stimulate PARP activity and oligonucleotide was not therefore required. Results were expressed in terms of pmol PAR former per mg tumour.

PARP Assay using Monoclonal Antibodies

As described in Plummer E R, Middleton M R, Jones C, Olsen A, Hickson I, McHugh P, Margison G, McGown G, Thorncroft M, Watson A J, Boddy A V, Calvert A H, Harris A L, Newell D R, Curtin N J. Temozolomide pharmacodynamics in patients with metastatic melanoma: DNA damage and activity of repair enzymes ATase and PARP-1. Clinical Cancer Research. 11 3402-3409 (2005) based on modification of previously published methods (Pfieffer R, Brabeck C, Burkle A: Quantitative nonisotopic immuno-dot-blot method for the assessment of cellular poly(ADP-ribosyl)ation capacity. Analytical Biochemistry 1999; 275:118-122).

Cultured cells or rapidly defrosted lymphocyte preparations were washed twice in ice cold PBS. The cell pellets were resuspended in 0.15 mg/ml digitonin to a density of approximately 1-2×10⁶ cells per ml for 5 minutes to permeabilise the cells, following which 9 volumes of ice-cold buffer (7 mM HEPES, 26 mM KCl, 0.1 mM dextran, 0.4 mM EGTA, 0.5 mM MgCl₂, 45 mM sucrose, pH 7.8) were added and the sample placed on ice. The permeabilised (i.e. trypan blue stained) cell density was counted and the cell suspension was diluted if necessary with the above buffer to achieve a cell density that allowed 20,000 permeabilised cells to be added to each reaction tube. In the assay maximally stimulated PARP activity is measured by exposure to a blunt ended oligonucleotide in the presence of NAD⁺ substrate [25], at 26° C. in an oscillating water bath. Five μl of 7 mM NAD⁺ and 5 μl 200 μg/ml pallindromic oligonucleotide (CGGMTTCCG) were mixed with permeabilised cells and reaction buffer (100 mM Tris HCl, 120 mM MgCl₂, pH 7.8) to a final volume of 100 μl. The reaction was stopped after 6 minutes by the addition of excess PARP inhibitor (400 μl of 12.5 μM Compound I) and the cells blotted onto a nitrocellulose membrane (Hybond N, Amersham) using a 24-well manifold. A purified PAR standard curve was loaded onto each membrane (0-25 pmol monomer equivalent) to allow quantification. Overnight incubation with the primary antibody (1 in 500 in PBS-MT (PBS plus 0.05% Tween 20 Plus 5% Milk powder) at 4° C. was followed by 2 washes in PBS-T (PBS plus 0.05% Tween 20) and then incubation in secondary antibody (1 in 1000 in PBS-MT) for 1 hour at room temperature. The incubated membrane was washed frequently with PBS over the course of one hour then exposed for one minute to ECL reaction solution as supplied by the manufacturer. Chemiluminesence detected during a 5 minute exposure was measured using a Fuji LAS3000 UV Illuminator (Raytek, Sheffield, UK) and digitised using the imaging software (Fuji LAS Image version 1.1, Raytek). The acquired image was analysed using Aida Image Analyser (version 3.28.001), and results expressed in LAU/mm². Three background areas on the exposed blot were measured and the mean of the background signal from the membrane subtracted from all results. The PAR polymer standard curve was analysed using an un-weighted one site binding non-linear regression model and unknowns read off the standard curve so generated. Results were then expressed relative to the number of cells loaded. Triplicate QC samples of 5000 L1210 cells were run with each assay, all samples from one patient being analysed on the same blot.

Tumour homogenates were assayed in a similar manner; however, the homogenisation process introduces sufficient DNA damage to maximally stimulate PARP activity and oligonucleotide was not therefore required. The protein concentration of the homogenate was measured using the BCA protein assay and Titertek Multiscan MCC/340 plate reader. Results can be expressed in terms of pmol PAR former per mg protein or per mg tumour.

The PARP activity assay in peripheral blood monocytes (PBMCs) is based on the method of Boulton et al. (“Potentiation of temozolomide-induced cytotoxicity: a comparative study of the biological effects of poly(ADP-ribose) polymerase inhibitors.” 1995. British J. Cancer 72, 849-856). All processes to be carried out at 0-4° C.

Preparation of PBMC's
1. Collect 5 ml blood into a Lithium Heparin tube and mix gently.
2. Dilute heparinised blood 1:1 with PBS in 30 ml disposable universal tube (final volume 10 ml).
3. Carefully layer the diluted blood over 8-10 ml of pre-chilled Lymphoprep in a 30 ml disposable universal tube. Take care not to mix the blood with the separation fluid.
4. Centrifuge the samples for 15 minutes in a swing out rotor (Mistral centrifuge) at 800×G, at 4° C., brake rate 0.
5. After centrifugation, a leukocyte band should be visible at the interface. This cell band should be harvested using a glass Pasteur pipette and put in a 30 ml disposable Universal tube.
6. Dilute the lymphocyte suspension with 20 ml ice cold PBS and centrifuge the cells for 10 minutes at 500×G, 4° C.
7. Remove the supernatant.
8. Resuspend the pellet in 20 ml ice cold PBS and centrifuge at 333×G/4° C. for 5 minutes.
9. Remove the supernatant and resuspend the cells in 500 μl pre-chilled medium (RPMI plus 10% foetal calf serum) supplemented with 10% DMSO
10. Transfer to a labelled screw capped Eppendorf tube and freeze.
11. Store at −70° C.

PARP Assay of PBMC's
1. ³²P 600 μM NAD solution is prepared fresh on the day of experiment as detailed above. Oligonucleotide stock is removed from storage and defrosted.
2. Water bath is warmed to 26° C. and set to agitate at 70 oscillations per minute.

3. Reaction test tubes are set up as follows.

| | | | | Final |
|---|---|---|---|---|
| Reagent | T0 | +oligo | −oligo | concentration |
| Oligonucleotide | 5 μl | 5 μl | | 2.5 μg/ml |
| ³²P 600 μM NAD stock | 50 μl | 50 μl | 50 μl | 75 μM |
| Water | 45 μl | 45 μl | 50 μl | |
| Running total | 100 μl | 100 μl | 100 μl | |
| Cell suspension | 300 μl | 300 μl | 300 μl | |
| Reaction Total | 400 μl | 400 μl | 400 μl | |

4. Each PBMC sample and a QC standard is assayed in triplicate, with T0, +oligo and −oligo samples ×3. (Total of 9 tubes per sample.)
5. The cell density in each suspension is calculated. A 10 μl sample of each cell suspension is diluted 1:1 with Trypan Blue and the number of permeabilised cells per ml counted on a haemocytometer.
6. Reaction test tubes and permeabilised cell suspension are warmed in the water bath to 26° C. for 7 minutes.
7. The permeabilised cell suspension is vortexed briefly and the reaction is started by adding 300 μl (approx. 1×10⁶ cells) of this to each reaction tube.
8. The reaction is stopped exactly 6 minutes after addition of cells by adding 2 ml of ice cold 10% TCA+10% NaPPi and vortexing.
9. The tube is then incubated on ice for at least one hour (at this stage of the assay precipitation must occur for at least one hour, the reaction tubes may be left overnight if the temperature is maintained at ≦4° C.) prior to filtration.
10. 2 ml ice cold 10% TCA+10% NaPPi is added to the T0 tubes prior to addition of permeabilised cells, to correct for non-specific binding of radio-label to the filter.

Preparation of Tumour/Tissue Samples
1. The frozen tumour samples are weighed.
2. 3 volumes (i.e. 3 μl solution added for each 1 mg tissue) of isotonic buffer plus DTT is added to the tumour sample. This is stored on ice until and during homogenisation.
3. The sample is homogenised on ice within a Class II cabinet for 10-second bursts until no detectable macroscopic pieces of tissue are visible.
4. Sufficient volume of the homogenate is diluted 1 in 10 with isotonic buffer plus DTT to provide an overall dilution of 1 in 40 from the original sample. A final volume of 3 ml is sufficient for triplicate sampling and a subsequent protein assay.
5. The diluted homogenate is stored on ice and assayed within one hour as below.

PARP Assay of Tumour/Tissue Samples
1. ³²P 600 μM NAD solution is prepared fresh on the day of experiment as detailed above. Oligonucleotide stock is removed from storage and defrosted.
2. Water bath is warmed to 26° C. and set to agitate at 70 oscillations per minute.

3. Reaction test tubes are set up as per table A for the QC samples and as per table B for the homogenate.

TABLE A

| | | | | Final |
|---|---|---|---|---|
| Reagent | T0 | +oligo | −oligo | concentration |
| Oligonucleotide | 5 μl | 5 μl | | 2.5 μg/ml |
| 600 μM [³²P] NAD⁺ stock | 50 μl | 50 μl | 50 μl | 75 μM |
| Water | 45 μl | 45 μl | 50 μl | |
| Running total | 100 μl | 100 μl | 100 μl | |
| Cell suspension | 300 μl | 300 μl | 300 μl | |
| Reaction Total | 400 μl | 400 μl | 400 μl | |

TABLE B

| Reagent | T0 | Reaction | Final concentration |
|---|---|---|---|
| 600 μM [³²P] NAD⁺ stock | 50 μl | 50 μl | 75 μM |
| Water | 50 μl | 50 μl | |
| Running total | 100 μl | 100 μl | |
| Homogenate | 300 μl | 300 μl | |
| Reaction Total | 400 μl | 400 μl | |

4. Each QC sample is assayed in triplicate, with T0, +oligo and −oligo samples ×3. (Total of 9 tubes per sample, if low cell counts −oligo samples are omitted.) Homogenates are also assayed in triplicate, with T0 and reaction samples ×3. (Total 6 tubes per sample).
5. 2 ml ice cold 10% TCA+10% NaPPi is added to the T0 tubes prior to addition of homogenate or cells, to correct for non-specific binding of radio-label to the filter.
6. Reaction test tubes homogenates and QC cells are warmed in the water bath to 26° C. for 7 minutes.
7. Each preparation is vortexed briefly and the reaction is started by adding 300 μl of this to each reaction tube.
8. The reaction is stopped exactly 6 minutes after addition of homogenate by adding 2 ml of ice cold 10% TCA+10% NaPPi and vortexing.
9. The tube is then incubated on ice for at least one hour prior to filtration.
10. A 10 μl sample of the QC suspension is diluted 1:1 with Trypan Blue and the number of permeabilised cells per ml counted on a haemocytometer.
11. The remaining homogenates are centrifuged at 500×G for 5 minutes at 4° C., 200 μl of the supernatant is removed and placed in a labelled screw capped microtube for protein measurement. Supernatant samples may be stored for at least one month at −20° C. if not assayed immediately.

### Example 1

### Inhibition of Poly-ADP-Ribose Polymerase

Crystallographic analysis of the compound of formula 1 bound to the inhibited target enzyme revealed that the drug binds to the active site of PARP-1, forming 3 hydrogen bonds. The PARP enzyme inhibiting activity of the compound of formula 1 was assayed as described in U.S. Pat. No. 6,495,541. The Kᵢ determined using ³²P-NAD⁺ incorporation into polymer by purified full-length human PARP-1, is 1.4 nM (Table 3). The compound of formula 1 is also a potent inhibitor of PARP-2 (Kᵢ=0.17 nM) and, based on strong structural similarities in the amino acid sequences among the various PARP family enzymes (tankyrase, V-PARP), the phosphate salt of the compound of formula 1 (Compound I) will likely bind with high affinity to these enzymes as well.

TABLE 3
Kinetic Constants for the Interaction of the Compound of
Formula 1 with PARP

| | PARP-1 Kᵢ | PARP-2 Kᵢ |
|---|---|---|
| Compound | (nM ± SD*) | (nM ± SD*) |
| compound of | 1.4 ± 0.2 | 0.17 ± 0.05 |
| formula 1 | | |

*SD = standard deviation.

### Example 2

### Inhibition of Cell Growth

The intrinsic growth inhibitory activity of the compound of formula 1 following 5-day continuous exposure (Table 4) was determined in A549, LoVo and SW620 cell lines as described in U.S. Pat. No. 6,495,541. GI₅₀ values (the concentration required to inhibit growth by 50%) ranged from 7 to 12 μM. Similarly, the ability of 0.4 μM the compound of formula 1 (ie, <5% of the IC₅₀) to increase the growth inhibitory potency of temozolomide and topotecan was determined (Table 2). The potentiation factor at the IC₅₀ concentration; PF₅₀, is calculated as: GI₅₀ temozolomide or topotecan alone/GI₅₀ temozolomide or topotecan+0.4 μM the compound of formula 1. There was an 8-fold decrease in the GI₅₀ of temozolomide in the LoVo cells and a 3.5-fold decrease in the GI₅₀ of temozolomide in A549 cells upon addition of 0.4 μM the compound of formula 1. There was a 1.6-fold decrease in the GI₅₀ of topotecan in the LoVo cells and a 2.6-fold decrease in the IC₅₀ of topotecan in both A549 and SW620 cells upon addition of 0.4 μM the compound of formula 1.

TABLE 4
Inhibition of Cell Growth by the Compound of Formula 1 and
Potentiation of Temozolomide and Topotecan by 0.4 μM the
compound of formula 1

| Cell line | A549 | LoVo | SW620 |
|---|---|---|---|
| GI₅₀ compound of formula 1 | 7 | 12 | 11 |
| (μM) | | | |
| Temozolomide PF₅₀ | 3.5 | 8.1 | — |
| Topotecan PF₅₀ | 1.6 | 1.7 | 2.6 |

### Example 3

### Chemosensitization of Standard Chemotherapeutic Agents by Compound I

In vitro studies of human tumor cells lines carried out according to the procedure described in U.S. Pat. No. 6,495,541 have shown that at sub-micromolar concentrations the compound of formula 1 enhances the sensitivity of cells to temozolomide and the type-1-topoisomerase inhibitors, topotecan and SN-38 (the active metabolite of irinotecan) against human H460 non-small cell lung cancer (NSCLC) cells (Table 5).

TABLE 5
Effect of the compound of formula 1 as a Glucuronate Salt on
the In Vitro Potency of Standard Chemotherapeutic Agents in
Human H460 NSCLC Cells

| Chemotherapeutic | | |
|---|---|---|
| Agent | Types | PF₅₀ (H460)^{a} |
| Paclitaxel | Microtubule antagonist | 0.77 |
| 5-Fluorouracil | Pyrimidine antagonist | 0.92 |
| Gemcitabine | Pyrimidine antagonist | 1.2 |
| 6-thioguanine | Purine antagonist | 1.1 |
| Doxorubicin | Anthracycline antibiotic | 1.1 |
| Oxaliplatin | Platinum compound | 0.98^{b} |
| Cisplatin | Platinum compound | 1.2 |
| Etoposide | Topoisomerase II inhibitor | 0.75^{b} |
| Topotecan | Topoisomerase I inhibitor | 1.6 |
| SN-38 | Topoisomerase I inhibitor | 2.2 |
| Temozolomide | Monofunctional methylating agent | 3.7^{b} |

^{a}PF₅₀ = GI₅₀ (Single Agent)/GI₅₀ (Agent +0.4 μM the compound of formula 1) in human H460 NSCLC cells.
^{b}Compound I (the phosphate salt of the compound of formula 1) was substituted for the compound of formula 1 glucuronate salt in these experiments.

### Example 4

### Inhibition of Cellular NAD Depletion and Poly-ADP-ribose Polymer Formation by the Compound of Formula 1

Poly(ADP_ribose) polymers and NAD⁺ were quantified as described by Abou-Ela et.al (Anal Biochem. (1988), 174:239-250) with minor modifications as follows. A549 cells (ATCC, Rockville, Md.) were seeded into 35 mm culture dishes and allowed to grow to confluence. Medium was removed and replaced with fresh medium containing 20-50 μCi ml⁻¹ [³H] adenine. Cells were labeled for 16 h at 37° C. Medium was replaced with fresh medium for 45 min prior to experimental manipulation. Following experimental manipulation, the medium was removed and the cells were rinsed with ice-cold phosphate buffered saline, pH 7.2, and harvested by the addition of 1 ml 20% ice-cold trichloroacetic acid. Acid insoluble material was removed from the dishes by scraping. The dishes were washed once with 1 ml 20% trichloroacetic acid and the samples were subjected to centrifugation. The supernatant was saved for NAD⁺ determination. The pellet was dissolved in 0.2 ml of ice-cold 98% formic acid, then diluted to 10 ml with ice cold deionized H₂O. Two hundred microliters of 10 mg/ml bovine serum albumin was added to facilitate precipitation. The concentration of trichloroacetic acid was adjust to 20% by addition of 2.55 ml 100% trichloroacetic acid. The acid insoluble fraction was collected by centrifugation.

NAD⁺ determination. The trichloroacetic acid supernatant was diluted to 10 ml with 250 mM ammonium formate, pH 8.6, and adjusted to pH 8.6 with concentrated ammonium hydroxide. The sample was applied to a 0.5 ml DHB-Sepharose column that had been pre-washed with 10 ml of 250 mM ammonium formate, pH 8.6. The column was washed with 10 ml 250 mM ammonium formate, pH 8.6 and 2 ml H₂O. NAD⁺ was eluted with 4 ml 250 mM ammonium formate, pH 4.5. Determination of ADP-ribose polymers. The acid-insoluble pellet was dissolved in 1 ml guanidinium chloride, 250 mM ammonium acetate, 10 mM EDTA, pH 6.0; and 1 ml of 1 M KOH, 100 mM EDTA. The sample was incubated at 37° C. for 2 h. The sample was diluted to 10 ml with 1 M guanidinium chloride, 250 mM ammonium acetate, 10 mM EDTA, pH 9.0 (buffer A), adjusted to pH 9.0 and applied to a 0.5 ml column of DHBB (Bio Rad) that had been pre-washed with 5 ml H₂O and 10 ml buffer A. Following application, the column was washed with 25 ml buffer A, followed by 10 ml 1 M ammonium bicarbonate, 1 mM EDTA, pH 9.0. Poly(ADP-ribose) was eluted with 0.5 ml H₂O. The ample was lyophilized to dryness, then suspended in 2 ml of 50 mM MOPS, 5 mM McGiₗₗ, pH 7.5. The suspension was digested by addition of 1 unit Snake venom phophodiesterase 1 (Worthington Biochemicals) and 1 unit BAP for 3 h at 37° C.

HPLC analysis: Analysis was performed by HPLC on 5 μm Beckman C18 ODS-reversed-phase column, with a 7 mM ammonium formate, 7% methanol running phase at a flow rate of 1 ml/min. Each sample was co-injected with 10 nmol each of adenosine and deoxyadenosine. One milliliter column fraction were collected and counted in 5 ml scintillation fluid.

In vitro potentiation of temozolomide by the compound of formula 1 correlates with inhibition of alkylating agent-induced cellular NAD depletion and blockage of poly-ADP-ribose polymer formation with an effective range of 5 to 400 nM. After DNA damage, cellular NAD is rapidly incorporated into poly-ADP-ribose polymer. The compound of formula 1, 5 nM (PF₅₀=1.3), greatly reduced MNNG-induced cellular NAD consumption and inhibited cellular poly-ADP-ribose formation by 89% (Table 6). The compound of formula 1 increases the potency of temozolomide in A549 cells by at least 2-fold at concentrations as low as 50 nM, corresponding to 93% inhibition of MNNG-induced NAD depletion and 95% inhibition of poly-ADP-ribose polymer formation. The compound of formula 1 also inhibits PARP catalytic activity, measured as inhibition of cellular NAD consumption in P388 mouse leukemia cells and mouse peripheral blood lymphocytes following activation of PARP by MNNG, hydrogen peroxide, or gamma irradiation (data not shown).

TABLE 6
Inhibition of PARP Activity by the compound of formula 1 in A549 Cells In Vitro

| | The compound | | | | |
|---|---|---|---|---|---|
| MNNG | of formula 1 | NAD | Polymer | PF₅₀ | PF₅₀ |
| Concentration | Concentration | (% DMSO^{a}) | (% MNNG^{b}) | (Topotecan) | (Temozolomide) |
| None^{a} | None | 100^{a} | 1 | | |
| 25 μM^{b} | None | 44 | 100^{b} | | |
| 25 μM | 0.0005 μM | 77 | 40 | 1.4 | 1 |
| 25 μM | 0.005 μM | 95 | 11 | 1.8 | 1.3 |
| 25 μM | 0.05 μM | 96 | 5 | 2.1 | 2.2 |
| 25 μM | 0.1 μM | 97 | 4 | 2.2 | 2.6 |
| 25 μM | 0.4 μM | 97 | 4 | 2.4 | 3.5 |

^{a}DMSO control.
^{b}MNNG only control.

### Example 5

### In vivo Antitumor Efficacy Studies for the Compound of Formula 1—Temozolomide

In vivo experiments were performed as described in Calabrese et.al (JNCI (2004), 96:56-67).

For these studies, the calculation of Compound I (the phosphate salt) dose and the glucuronate salt dose was based on the free base.

In these studies, Compound I demonstrated no single-agent antitumor effects. In combination studies Compound I increased the dose potency of gamma irradiation, irinotecan, and temozolomide. In a single-dose experiment the compound of formula 1 enhanced the antitumor effects of 200 mg/kg temozolomide over a 10-fold range of nontoxic dosages in the SW620 human colon carcinoma xenograft in mice (Table 7).

TABLE 7
In Vivo Activity of the Compound of Formula 1 in Combination with
a Single Dose of Temozolomide Against the Human SW620 Colon
Carcinoma Xenograft

| | | Dose | | |
|---|---|---|---|---|
| Tumor | Dose | compound of | | Enhancement |
| model | Temozolomide^{a} | formula 1^{b} | Regimen^{c} | (%)^{d} |
| SW620 | 200 mg/kg | 0.1 mg/kg | Single dose | 50^{e} |
| SW620 | 200 mg/kg | 0.3 mg/kg | Single dose | 80^{e} |
| SW620 | 200 mg/kg | 1 mg/kg | Single dose | 107^{e} |
| SW620 | 200 mg/kg | 5 mg/kg | Single dose | 111^{e,f} |

^{a}Temozolomide dosage was delivered by oral gavage.
^{b}compound of formula 1 dosage was delivered by intraperitoneal injection.
^{c}n = 5 for all groups.
^{d}% Enhancement = 100 × (Delay with temozolomide + compound of Formula 1 − Delay temozolomide alone)/Delay temozolomide alone. Delay is calculated as time to RTV(relative tumor volume)4 in treated group - time to RTV4 in controls, where RTV4 is the tumor volume equivalent to 4x the tumor volume at the start of treatment.
^{e}Significantly different from single agent temozolomide (Mann-Whitney test)
^{f}2 deaths due to toxicity.

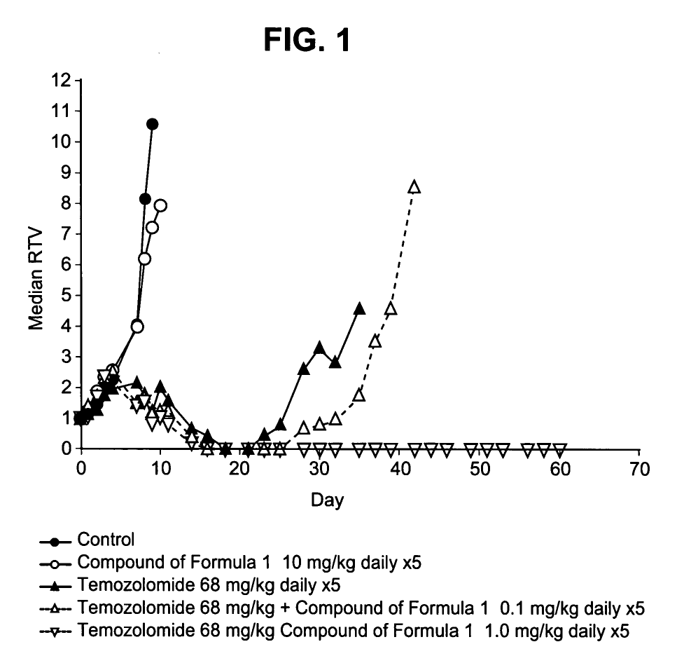

In a repeat-dose experiment (QD×5 for each agent) against the SW620 xenograft, the compound of formula 1 (as the glucuronate salt) at 0.05, 0.15, and 0.5 mg/kg in combination with 68 mg/kg temozolomide enhanced the activity of temozolomide in all 3 combination groups (68 mg/kg temozolomide) +0.05, 0.15, or 0.5 mg/kg the compound of formula 1 glucuronate salt. A 100% complete remission rate was observed in the combination groups with 0.15 or 0.5 mg/kg the compound of formula 1 glucuronate salt. No body weight loss was observed with the compound of formula 1 glucuronate salt dose combinations (68 mg/kg temozolomide +0.05 or 0.15 mg/kg the compound of formula 1 glucuronate salt). One toxic mortality was observed with the high-dose combination group (68 mg/kg temozolomide +0.5 mg/kg the compound of formula 1 glucuronate salt). In a similar experiment against LoVo xenografts, the compound of formula 1 glucuronate salt (0.5 mg/kg) enhanced the antitumor activity of temozolomide (68 mg/kg) by 67% (Table 8, FIG. 1). No body weight loss was observed in any dose group in the LoVo experiment. In no combination study was antagonism observed.

TABLE 8
Efficacy of Temozolomide in Combination With The Compound Of
Formula 1 As The Glucuronate Salt Against SW620 and LoVo Xenografts

| | | | | En- |
|---|---|---|---|---|
| | | Dose the | | hance- |
| Tumor | Dose | compound of | | ment^{e} |
| model | Temozolomide^{a} | formula 1^{b,c} | Regimen^{d} | (%) |
| SW620 | 68 mg/kg | 0.05 mg/kg | Once daily for 5 days | 35 |
| SW620 | 68 mg/kg | 0.15 mg/kg | Once daily for 5 days | 270^{f} |
| SW620 | 68 mg/kg | 0.5 mg/kg | Once daily for 5 days | >60^{f, g} |
| LoVo | 68 mg/kg | 0.05 mg/kg | Once daily for 5 days | — |
| LoVo | 68 mg/kg | 0.15 mg/kg | Once daily for 5 days | — |
| LoVo | 68 mg/kg | 0.5 mg/kg | Once daily for 5 days | 67^{f} |

^{a}Temozolomide dosages were delivered by oral gavage.
^{b}the compound of formula 1 dosages were delivered by intraperitoneal injection.
^{c}the compound of formula 1 (free base equivalent) dosed as glucuronate salt
^{d}n = 5 for all groups.
^{e}% Enhancement = 100 × (Delay with temozolomide + compound of Formula 1 − Delay temozolomide alone)/Delay temozolomide alone. Delay is calculated as time to RTV(relative tumor volume)4 in treated group − time to RTV4 in controls, where RTV4 is the tumor volume equivalent to 4x the tumor volume at the start of treatment.
^{f}Significantly different from single agent temozolomide (Mann-Whitney test)
^{g}1 mortality due to toxicity.

### Example 7

### In vivo Antitumor Efficacy Studies for the Compound of Formula 1—Irinotecan

In vivo experiments were performed as described in Calabrese et.al (J. Natl. Cancer Inst. (2004), 96:56-67).

As a single agent the topoisomerase I inhibitor, irinotecan (25 mg/kg, QW×3 IP), did not significantly inhibit SW620 tumor growth. The combination of 25 mg/kg irinotecan with the compound of formula 1 dosed as the glucuronate salt resulted in substantial antitumor effects and enhancement of irinotecan activity in all combination groups (25 mg/kg irinotecan+0.05, 0.15, or 0.5 mg/kg the compound of formula 1 (Table 9). No significant toxicity was observed in the irinotecan single agent groups or in the groups with irinotecan and PARP inhibitor combined. Tumor growth inhibition (percent enhancement) increased with increasing dosages of the compound of formula 1. In a similar experiment against LoVo xenografts the compound of formula 1 (0.5 mg/kg) enhanced the antitumor activity of irinotecan (25 mg/kg) by 86% (Table 9). In no combination study was antagonism observed.

TABLE 9
Efficacy of Irinotecan in Combination With The Compound Of Formula 1
as the Glucuronate Salt Against SW620 and LoVo Xenografts

| | | Dose the | | |
|---|---|---|---|---|
| | | compound of | | |
| Tumor | Dose | formula | | Enhancement^{d,e} |
| model | Irinotecan^{a} | 1 GS^{b,f} | Regimen^{c} | (%) |
| SW620 | 25 mg/kg | 0.05 mg/kg | Once weekly × 3 | 700 |
| SW620 | 25 mg/kg | 0.15 mg/kg | Once weekly × 3 | 1100^{f} |
| SW620 | 25 mg/kg | 0.5 mg/kg | Once weekly × 3 | 1100^{f} |
| LoVo | 25 mg/kg | 0.0 mg/kg | Once weekly × 3 | 0 |
| LoVo | 25 mg/kg | 0.15 mg/kg | Once weekly × 3 | 71 |
| LoVo | 25 mg/kg | 0.5 mg/kg | Once weekly × 3 | 86^{f} |

^{a}Irinotecan dosages were delivered by intraperitoneal injection.
^{b}the compound of formula 1 glucuronate salt dosages were delivered by intraperitoneal injection.
^{c}n = 5 for all groups.
^{d}% Enhancement = 100 × (Delay with irinotecan + compound of Formula 1 − Delay irinotecan alone)/Delay irinotecan alone. Delay is calculated as time to RTV(relative tumor volume)4 in treated group − time to RTV4 in controls, where RTV4 is the tumor volume equivalent to 4x the tumor volume at the start of treatment.
^{e}the compound of formula 1 (free base equivalent) dosed as glucuronate salt.
^{f}Significantly different from single agent irinotecan (Mann-Whitney test)

### Example 8

### Pharmacodynamics of the Compound of Formula 1—Temozolomide

Treatment of mice bearing the SW620 human colon carcinoma xenograft with 10 mg/kg the compound of formula 1 alone (QD×5) resulted in no tumor growth delay and was not toxic. Table 10(a) represents plasma and tumor concentration of the compound of formula 1 after intraperitoneal administration of the phosphate salt (Compound I). In a repeat-dose combination experiment (QD×5 for each agent) against the SW620 xenograft, 0.1 mg/kg the compound of formula 1 enhanced the antitumor effects of 68 mg/kg temozolomide by 28% over that of 68 or 136 mg/kg temozolomide alone (Table 10(b)). Increasing the dosage of the compound of formula 1 to 1 mg/kg enhanced the antitumor effects of temozolomide (68 mg/kg) by 100%. The combination of 10 mg/kg the compound of formula 1 and 68 mg/kg temozolomide was toxic.

In a parallel study plasma and tumor levels of the compound of formula 1 were measured by an HPLC/MS assay. In addition the degree of inhibition of tumor PARP catalytic activity was assessed using P³²-NAD incorporation into poly-ADP-ribose polymer in homogenates from SW620 tumors of treated animals. At the effective dosage of the compound of formula 1 (1.0 mg/kg), the compound of formula 1 plasma concentrations were barely detectable at 6 hours but tumor levels of 40 to 60 ng/mL were detectable at 6 and 24 h after injection. PARP catalytic activity was inhibited by 50% at 6 h and by 25% at 24 h.

At the toxic dosage of the compound of formula 1 (10 mg/kg), the compound of formula 1 plasma concentrations were 30 ng/mL at 6 h but barely detectable at 24 h. Tumor levels of the compound of formula 1 >200 ng/mL were detectable at all times up to 24 h after a dosage of 10 mg/kg of the compound of formula 1 and PARP catalytic activity was inhibited by 90% at 6 h and by 75% at 24 h.

TABLE 10(a)
Plasma and Tumor Concentration of the Compound of Formula 1
after Intraperitoneal Administration of the Phosphate Salt (Compound I)

| Dose | | Plasma | Tumor |
|---|---|---|---|
| compound | | compound | compound |
| of formula 1^{a,b} | Time | of formula 1 | of formula 1 |
| (mg/kg) | (h) | (ng/mL ± SD^{e}) | (ng/gm ± SD^{e}) |
| 1.0 | 0.5 | 107 ± 32.3 | 98.3 ± 25.2 |
| 1.0 | 6 | 6.64 ± 1.55 | 49.5 ± 2.12 |
| 1.0 | 24 | 1.85 ± 0.14 | BLQ^{c} |
| 10 | 0.5 | 1867 + 102 | 767 ± 55.6 |
| 10 | 6 | 64.4 ± 11 | 523 ± 37.3 |
| 10 | 24 | 2.34 ± 0.33 | 167 ± 54.2 |

^{a}the compound of formula 1 dosages were delivered by intraperitoneal injection.
^{b}the compound of formula 1 (free base equivalent) dosed as phosphate salt.
^{c}BLQ: Below limit of quantitiation

TABLE 10(b)
Efficacy and Pharmacodynamics of the Compound of Formula 1 as
the Glucuronate Salt in Combination with Temozolomide Against the
SW620 Human Colon Carcinoma Xenograft

| Dose | | Plasma | | |
|---|---|---|---|---|
| compound of | | compound of | | |
| formula 1^{a,b,d} | Time | formula 1 | PARP | En- |
| (mg/kg) | (h) | (ng/mL ± SD^{e}) | Activity (%) | hancement^{f} (%) |
| Control | | | 100 | |
| Temozolomide | | | 100 | |
| alone^{c} | | | | |
| 0.1 | 0.5 | 12.7 ± 2.3 | 77.6 ± 13.3 | 28^{g} |
| 0.1 | 6 | 0.0 ± 0.0 | 90.2 ± 11.5 | |
| 0.1 | 24 | 0.0 ± 0.0 | 100.4 ± 7.5 | |
| 1.0 | 0.5 | 92.0 ± 28.0 | 24.9 ± 16.2 | ≧100^{g} |
| 1.0 | 6 | 4.7 ± 4.0 | 68.6 ± 36.2 | |
| 1.0 | 24 | 0.0 ± 0.0 | 70.2 ± 19.0 | |
| 10 | 0.5 | 1532 + 134 | 3.4 ± 1.4 | N/A^{h} |
| 10 | 6 | 98.0 ± 28.0 | 4.8 ± 0.87 | |
| 10 | 24 | 17.8 ± 29.5 | 18.3 ± 12.7 | |

^{a}the compound of formula 1 dosages were delivered by intraperitoneal injection.
^{b}the compound of formula 1 (free base equivalent) dosed as glucuronate salt.
^{c}Temozolomide dosages were delivered by oral gavage.
^{d}Dosage of the compound of formula 1, delivered i.p., in combination with 68 mg/kg Temozolomide, delivered p.o.
^{e}SD = standard deviation.
^{f}Enhancement calculated as ((Delay (combination)/Delay (temozolomide alone)) × 100 − 100)
^{g}Significantly different from single agent temozolomide (Mann-Whitney test)
^{h}N/A, not applicable, 5/5 mortalities due to toxicity.

### Example 9

### Pharmacokinetic Studies in Animals

The compound of formula 1 (free base drug substance) pharmacokinetics, following IV administration of the compound of formula 1 salts, were evaluated in CD-1 mice, Wistar rats, Beagle dogs, and cynomolgus monkeys and is summarized in Table 11. IV dosing to all species resulted in moderate to rapid clearance (34 to 136 mL/min/kg) and a large volume of distribution (7 to 15 L/kg), indicating this compound is well distributed in the body. The terminal half-life was relatively short to moderate (2 to 5 hours). Combination studies of Compound I (the phosphate salt) with temozolomide were conducted in mice and rats to investigate the potential impact of this cytotoxic agent on the pharmacokinetics of the compound of formula 1. For the mouse combination study, one group of 8 mice received a single 6.5 mg/kg IV dose of Compound I (equivalent to 5 mg/kg of the compound of formula 1) while a second group of 8 mice received a single 6.5 mg/kg IV dose of Compound 1 and a single 200 mg/kg oral dose of temozolomide. Each of the dose treatment groups was split into 2 cohort groups of 4 mice. The reason for cohort blood sampling is due to the blood volume limitations of the mouse species. Blood was drawn from each cohort every other pharmacokinetic sampling time. For the rat combination study, one group of 2 rats received a single 6.5 mg/kg IV dose of Compound 1 (5 mg/kg) while a second group of 2 rats received both the 6.5 mg/kg IV dose of Compound I and a 50 mg/kg oral dose of temozolomide. Results from the combination study of Compound I and temozolomide in mice and rats showed this cytotoxic agent to have only minor effects on the pharmacokinetic profile of the compound of formula 1 (Table 12 and Table 13). Similarly, Compound I was shown to have only minor effects on the pharmacokinetic profile of temozolomide (data not shown). In addition, combination studies of Compound I and irinotecan were conducted in male CD-1 mice and male Wistar rats. For the mouse combination study, one group of 15 mice received a single 6.5 mg/kg IV dose of Compound I (equivalent to 5 mg/kg of the compound of formula 1) while a second group received both a 6.5 mg/kg IV dose of Compound I and a 45 mg/kg IV dose of irinotecan. Three mice per dose group were euthanized at each of the collection time points. For the rat combination study, one group received a 6.5 mg/kg IV dose of Compound I while the second group received both the 6.5 mg/kg IV dose of Compound I and the 45 mg/kg dose of irinotecan. Blood from each rat was collected for each time point. Results from this study suggest that at the administered doses there are no drug-drug interactions between Compound I and irinotecan that result in altered pharmacokinetics (Table 14 and Table 15).

TABLE 11
Mean Pharmacokinetic Parameters of a Single IV Dose
of the Compound of Formula 1 in Mice, Rats, Dogs, and Monkeys

| | Dose | Vₛₛ | CL | t_{1/2} | AUC_{(0-∞)} |
|---|---|---|---|---|---|
| Species | (mg/kg) | (L/kg) | (mL/min/kg) | (hours) | (μg · h/mL) |
| Mouse^{a} | 5^{b} | 10 | 136 | 2.3 | 0.62 |
| Rat^{a} | 5^{b} | 10 | 85 | 2.8 | 0.99 |
| Dog | 15^{c} | 15.2 ± 2.3 | 61.8 ± 8.2 | 4.5 ± 1.1 | 4.1 ± 0.5 |
| Monkey | 15^{c} | 7.2 ± 1.2 | 33.8 ± 3.1 | 5.2 ± 0.8 | 7.4 ± 0.7 |

Group mean from n = 15 mice, all others n = 2 or 3 (± SD)
^{a}Mouse and rat data are from the combination studies with temozolomide
^{b}the compound of formula 1 (free base equivalent) dosed as Compound I (phosphate salt)
^{c}the compound of formula 1 (free base equivalent) dosed as glucuronate salt

TABLE 12
Group Mean the Compound of Formula 1 Pharmacokinetic Parameters
in Mice Dosed with Compound I (phosphate salt) Alone or in Combination
with Temozolomide

| | | Compound I |
|---|---|---|
| | Compound I | (+TEMO) |
| Route of Administration | IV | IV (Oral) |
| Dose (mg/kg)^{a} | 5 | 5 (200) |
| AUC_{(0-∞)} (μg · h/mL) | 0.62 | 0.95 |
| CL (mL/min/kg) | 136 | 88 |
| Vₛₛ (L/kg) | 10 | 9 |
| t_{1/2} (hours) | 2.3 | 2.2 |

^{a}Compound I (phosphate salt of the compound of formula 1); doses corrected for salt.

TABLE 13
Mean the Compound of Formula 1 Pharmacokinetic Parameters
in Rats Dosed with Compound I (phosphate salt) Alone or in Combination
with Temozolomide

| | | Compound I |
|---|---|---|
| | Compound I | (+TEMO) |
| Route of Administration | IV | IV (Oral) |
| Dose (mg/kg)^{a} | 5 | 5 (50) |
| AUC_{(0-∞)} (μg · h/mL) | 1.0 | 0.7 |
| CL (mL/min/kg) | 85 | 123 |
| Vₛₛ (L/kg) | 10 | 11 |
| t_{1/2} (hours) | 2.8 | 1.6 |

^{a}Compound I (phosphate salt of the compound of formula 1); doses corrected for salt.

TABLE 14
Group Mean the Compound of Formula 1 IV Pharmacokinetic Parameters
in Mice Dosed with Compound I (phosphate salt) Alone or in Combination
with Irinotecan

| | | Compound I |
|---|---|---|
| | Compound I | (+IRINO) |
| Route of Administration | IV | IV (IV) |
| Dose (mg/kg)^{a} | 5 | 5 (45) |
| AUC_{(0-∞)} (μg · h/mL) | 0.93 | 1.12 |
| CL (mL/min/kg) | 90 | 75 |
| Vₛₛ (L/kg) | 11 | 6 |
| t_{1/2} (h) | 2.1 | 1.6 |

^{a}Compound I (phosphate salt of the compound of formula 1); doses corrected for salt.

TABLE 15
Mean (SD) the Compound of Formula 1 IV Pharmacokinetic Parameters
in Rats Dosed with Compound I (phosphate salt) Alone or in Combination
with Irinotecan

| | | Compound I |
|---|---|---|
| | Compound I | (+IRINO) |
| Route of Administration | IV | IV (IV) |
| Dose (mg/kg)^{a} | 5 | 5 (45) |
| AUC_{(0-∞)} (μg · h/mL) | 0.70 (0.06) | 0.91 (0.03) |
| CL (mL/min/kg) | 119 (10.99) | 91 (2.87) |
| Vₛₛ (L/kg) | 16 (4.13) | 14 (0.88) |
| t_{1/2} (h) | 2.2 (0.28) | 2.3 (0.07) |

^{a}Compound I (phosphate salt of the compound of formula 1); doses corrected for salt.

### Example 10

### Effects in Humans: a Phase 1 Trial of the Intravenous PARP Inhibitor Compound I in Combination with Five Days of Oral Temozolomide Given Every Four Weeks

This is an open-label, multi-center, dose-escalation study being conducted in 2 parts. Part 1 of the study was open to patients with advanced tumors. Following a test dose of single-agent Compound I given on Day −7, Compound I was given as a daily IV infusion for 5 days with temozolomide (100 mg/m²/dose). In sequential cohorts of patients, doses of Compound I were escalated until the PARP inhibitory dose (PID, see section D below) was identified by pharmacodynamic and pharmacokinetic data. The PID has been determined to be 12 mg/m². Intrapatient escalation of Compound I was allowed after safety of the higher dose was established in a previous cohort.

Part 2 of the study is open to patients with metastatic melanoma. Sequential cohorts of patients receive the PID of Compound I in addition to escalating doses of temozolomide until the MTD of the combined drugs is established or the temozolomide dose reaches a maximum of 200 mg/m². Patients entering part 2 of the study must consent to a pre- and posttreatment tumor biopsy to measure PARP inhibition. Intrapatient escalation of temozolomide is allowed after safety of the higher dose is established in a previous cohort.

Clinical results on 17 patients were consented and treated in part 1 of study. Table 16 shows the demographics of these patients.

TABLE 16
Patients Demographics in Part 1 of Phase 1 Study

| | | | | | Performance | Median Body |
|---|---|---|---|---|---|---|
| Cohort ID | Compound I | Median | | Ethnic | Status | Surface Area |
| (# | Dose | Age (yr) | Sex | Origin | (WHO) | (m²) |
| Patients) | (mg/m²) | (Range) | % Male | % White | 0/1% | (Range) |
| 1 (3) | 1 | 56 (49-62) | 33 | 100 | 67/33 | 1.74 (1.32-1.82) |
| 2 (4) | 2 | 55 (32-72) | 100 | 100 | 75/25 | 2.29 (1.86-2.44) |
| 3 (3) | 4 | 55 (36-56) | 67 | 100 | 0/100 | 1.80 (1.71-2.01) |
| 4 (4) | 8 | 55 (31-68) | 75 | 100 | 25/75 | 1.94 (1.82-2.10) |
| 5 (3) | 12 | 65 (59-71) | 100 | 100 | 33/67 | 2.06 (2.04-2.26) |
| Total (17) | | 56 (31-72) | 76 | 100 | 41/59 | 2.01 (1.32-2.44) |

Primary cancer diagnoses of these patients, all with advanced disease, were breast (1), colon (2), kidney (1), liver (1), pancreas (2), prostate (1), rectum (1), melanoma (3), soft tissue sarcoma (3), and stomach (2). Twelve (71%) patients received prior chemotherapy, 3 (18%) patients did not, and 2 (12%) have no information.

A. Pharmacokinetics and Product Metabolism in Humans

The pharmacokinetics of the compound of formula 1 was evaluated in the Phase 1 open-label, dose-escalation study of IV Compound I in combination with temozolomide. In part 1 of the study (dose escalation of Compound I), serial blood samples were collected for the compound of formula 1 determination at the following times:
Cycle 1, Day −7 (C1D-7, Compound I single dose)
Cycle 1, Day 1 (C1D1, Compound I plus temozolomide single dose)
Cycle 1, Day 4 (C1D4, Compound I plus temozolomide multiple dose)

The PK analysis was conducted on preliminary interim data using nominal collection times.

B. PK Analysis at All Cycles up to Day 4

Determination of the compound of formula 1 in human plasma was performed by using protein precipitation extraction followed by reverse phase HPLC with tandem mass spectrometric detection. The following chromatographic conditions were used:
Analytical Column: Thermo Hypersil Keystone Betabasic C8, 5 μm, 100×2.1 mm ID
Mobile Phase A Composition: 0.1% formic acid in water
Mobile Phase B Composition: 0.1% formic acid in acetonitrile
Flow Rate: 200 μL/min
Injection Volume: 10 μL
Autosampler Needle Wash: Water: acetonitrile: formic acid (500:500:1, v:v:v)
Typical Retention Times*: Compound of formula 1:1.5 minutes d₆-Compound of formula 1:1.5 minutes
*Retention times are approximate, and may vary between and within analytical batches.
The following are typical mass spectrometry parameters and may vary between instruments to obtain the equivalent response:
Mass Spectrometer: Sciex API 365
Ionisation: Sciex Turbo Ion Spray
Turbo Ionspray: positive ion mode
Ion Spray Voltage: 4000 V
Turbo Heater Temperature: 450° C.
Mass Transitions (nominal): Compound of formula 1: m/z=324.4→m/z 293.2 d₆-Compound of formula 1: m/z=330.3→m/z 299.1
Dwell Time: Compound of formula 1: 350 ms d₆-Compound of formula 1: 150 ms
Nebuliser Gas Pressure: 6
Curtain Gas Setting: 8

CAD: 2

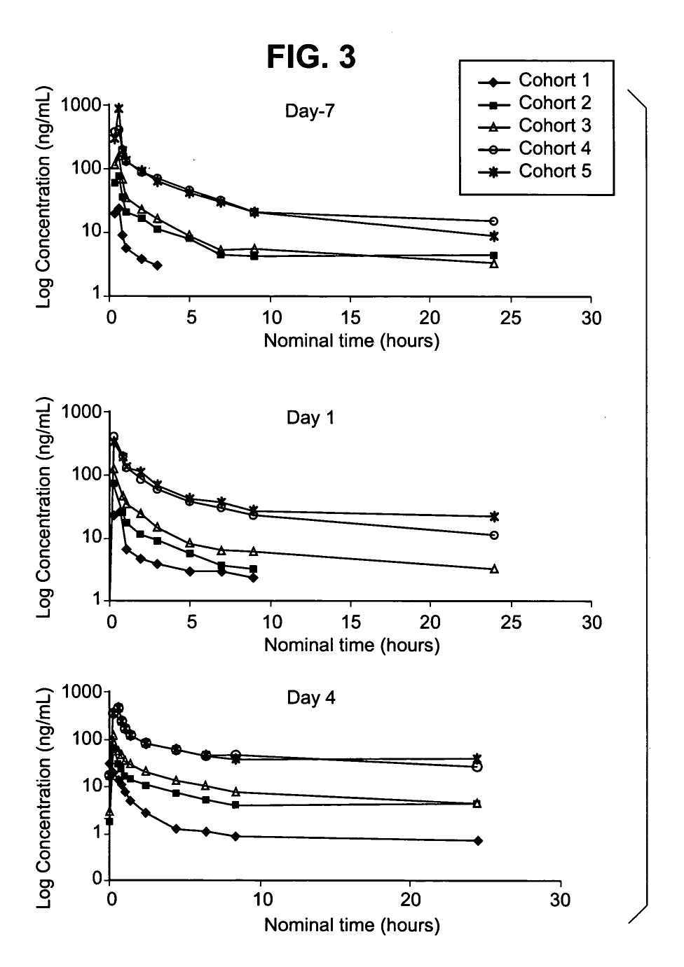

A summary of the preliminary PK parameters in all 17 patients is presented in Table 17 and the mean plasma concentration-time profiles of the compound of formula 1 for each dose cohort are shown in FIG. 3.

TABLE 17
Summary of Preliminary Pharmacokinetic Parameters (Mean (CV %)) of the compound of
formula 1 Following a 30-minute IV Infusion of Compound I Alone (C1D-7), or Compound I
Plus Oral 100 mg/m² Temozolomide (C1D1 and C1D4)

| | Com- | | | Cmax | | AUC₀₋₂₄ | | AUC_{inf}^{a} | | Vss | | CL^{a} | | t_{1/2} | |
|---|---|---|---|---|---|---|---|---|---|---|---|---|---|---|---|
| | pound I | temo | | ng/mL | | (ng * h/mL) | | (ng * h/mL) | | (L) | | (L/hr) | | (h) | |
| cohort | dose | dose | | mean | cv % | mean | cv % | mean | cv % | mean | cv % | mean | cv % | mean | cv % |
| 1 | 1 mg/m² | 100 | day −7 | 24.8 | 23 | 24.2 | 39 | 24.2 | 39 | 72.9^{b} | 38 | 72.4^{b} | 28 | 1.2^{b} | 56 |
| | (n = 3) | | day 1 | 26.5 | 7 | 37.5 | 67 | 39.6 | 74 | 114.3^{b} | 87 | 54^{b} | 56 | 3.5^{b} | 123 |
| | | | day 4 | 26.9 | 27 | 46.8 | 85 | — | — | 227.0^{b} | 118 | ^{49.2}^{b} | 52 | 9.3^{b} | 148 |
| 2 | 2 mg/m² | 100 | day −7 | 71.6 | 21 | 139.0 | 54 | 167.0 | 78 | 158.8 | 53 | 34.9 | 42 | 6.2 | 116 |
| | (n = 3-4) | | day 1 | 84.9 | 15 | 106.9 | 14 | 108.1 | 14 | 138.8 | 14 | 39.6 | 3 | 3.7 | 17 |
| | | | day 4 | 74.1 | 16 | 159.6 | 39 | — | — | 259.9 | 36 | 29.7 | 38 | 7.7 | 76 |
| 3 | 4 mg/m² | 100 | day −7 | 168.9 | 61 | 237.3 | 20 | 286.6 | 18 | 286.4 | 94 | 26.0 | 15 | 10.7 | 69 |
| | (n = 3) | | day 1 | 133.8 | 18 | 212.8 | 29 | 255.9 | 40 | 241.4 | 68 | 32.1 | 42 | 9.8 | 69 |
| | | | day 4 | 159.0 | 18 | 284.9 | 24 | — | — | 214.1 | 34 | 26.7 | 23 | 9.5 | 55 |
| 4 | 8 mg/m² | 100 | day −7 | 456.4 | 24 | 892.4 | 36 | 1118.8 | 45 | 151.7 | 35 | 16.9 | 54 | 10.5 | 55 |
| | (n = 4) | | day 1 | 472.8 | 33 | 883.0 | 29 | 1105.8 | 25 | 203.2 | 34 | 14.7 | 24 | 13.5 | 22 |
| | | | day 4 | 558.6 | 54 | 1363.9 | 39 | — | — | 230.5 | 21 | 12.8 | 38 | 17.9 | 23 |
| 5 | 12 mg/m² | 100-200 | day −7 | 882.5 | 95 | 1017.1 | 38 | 1163.3 | 36 | 270.1 | 8 | 23.9 | 38 | 10.6 | 24 |
| | (n = 3) | | day 1 | 867.9 | 68 | 1159.6 | 39 | 1475.9 | 44 | 278.4 | 3 | 19.0 | 32 | 11.5 | 26 |
| | | | day 4 | 670.5 | 24 | 1718.0 | 36 | — | — | 250.7 | 15 | 10.1 | 45 | 22.1 | 31 |

^{a}AUC_{0-inf} and CL may not be reflected accurately as the extrapolation for AUC_{0-inf} was >20% of the AUC₀₋₂₄ for some patients.
^{b}Not included in statistical analysis. The value may not be correctly estimated due to insufficient data.

B.1. PK Analysis at Cycle 1 Day-7 (C1D-7)

After IV infusion of Compound I alone for 30 minutes (C1D-7), the plasma concentrations of the compound of formula 1 declined in a multi-exponential manner with a mean terminal half-life of about 6.2-10.7 hours. Between 2 to 12 mg/m² of Compound I alone given as a 30-minute IV infusion, there was linear dose proportionality in AUC₍₀₋₂₄₎ and Cₘₐₓ. The AUC₀₋₂₄ at 1 mg/m² was not included in the evaluation of dose proportionality because the concentrations were below the limit of the analytical assay (LLOQ=2 ng/mL) for all patients after 3 hours of dosing. The mean total body clearance was 27 L/h (C1D-7), which is approximately 30% of hepatic blood flow. The mean steady state volume of distribution was 197 L (C1D-7).

B.2. PK Analysis at Cycle 1 Day 1 (C1D1)

After a single oral dose of 100 mg/m² temozolomide and single doses of 1 to 12 mg/m² of Compound I, the compound of formula 1 concentrations were similar to those of Compound I given alone. The compound of formula 1 AUC₍₀₋₂₄₎ on C1D-7 (Compound I alone) was comparable with that on C1D1 (Compound I plus temozolomide) at all doses.

B.3. PK Analysis at Cycle 1 Day 4 (C1D4)

After 4 days of daily dosing of Compound I plus temozolomide, there was minimal accumulation of the compound of formula 1 in plasma based on visual inspection of the individual plasma concentration-time profiles. However, there was a trend of increasing (range: 50% to 75%) the compound of formula 1 AUC₍₀₋₂₄₎ between the dose on Cycle 1 Day 4 and the dose on Cycle 1 Day 1 (Table 17).

B.4. Inter- and Intra-Patient Variability

The compound of formula 1 interpatient variability of AUC₍₀₋₂₄₎ was 14% to 85% and of CMU was 7% to 95%. However, interpatient variability within each cohort was <60% in general for both AUC₍₀₋₂₄₎ and Cₘₐₓ (Table 17). The intrapatient variability of AUC₍₀₋₂₄₎ and C. was assessed by comparing Compound I alone on C1D-7 with Compound I+temozolomide on C1D1. Intrapatient variability ranged from 7% to 47% for AUC₍₀₋₂₄₎ and 3% to 44% for Cₘₐₓ.

C. Determination of PARP Inhibitory Dose: Assay Methodology

A pharmacodynamic assay for PARP activity and inhibition uses monoclonal antibodies to measure the amount of PAR polymer that is formed under set conditions in permeabilized peripheral blood lymphocytes and homogenized tumor samples. The quantity of polymer formed can be used as a correlate for PARP activity, whereby decreasing polymer formation correlates with degree of PARP inhibition. PARP activity is expressed as a percentage of baseline, and is calculated by dividing the amount of PAR polymer formed after infusion by the quantity formed before infusion. The feasibility of this assay was successfully tested in the 12 patients Phase 2 study of single-agent temozolomide in patients with metastatic melanoma. This study showed that single-agent temozolomide did not inhibit PARP activity in either peripheral blood lymphocytes or tumor biopsy specimens.

D. Pharmacodynamics Evaluated from Phase 1 Clinical Study

In the Phase 1 open-label, dose-escalation study of IV Compound I in combination with temozolomide, one of the primary objectives of the study was to determine the PID of Compound I; the PID was defined as the dose at which PARP activity in peripheral blood lymphocytes was reduced to less than 50% of baseline, and there was a plateau (±10% absolute) in the degree of PARP inhibition between 2 Compound I dose levels. The definition was based on PARP activity observed 24 hours after administration of Compound I on Day 1.

Using a pharmacodynamic assay for PARP activity disclosed in Section C above, PARP inhibition in peripheral blood lymphocytes and tumor tissue was assessed in a Phase 1 clinical trial. As indicated above, enrollment in the Phase 1 Part 2 study has been restricted to patients having metastatic melanoma with biopsiable disease. All patients have been required to consent to pre-treatment and post-treatment biopsies such that PARP activity in the tumor can be evaluated.

In the Phase 1 study, whole blood samples were collected from all patients on the day that the test dose of Compound I was administered (usually Day −7), and on Days 1 and 4. The timing of the collections was before infusion of Compound I, end of infusion, 4 to 6 hours after infusion, and 24 hours after infusion (before infusion next day). Compound I was administered by IV infusion over 30 minutes. Peripheral blood lymphocytes were harvested from the blood samples, and where possible, the samples were analyzed in triplicate.

In Table 18 PARP activity in peripheral blood lymphocytes following administration of Compound I on Day 1 is summarized. Marked PARP inhibition (at least a 50% decrease in median PARP activity) was shown in all patients regardless of dose after completion of the 30-minute infusion of Compound I on Day 1. Depending on the patient, maximum inhibition was observed at the 0.5 or 4- to 6-hour time point. Durable inhibition of PARP activity was demonstrated in all patients in cohorts 4 and 5, where >50% median reduction in PARP activity was observed 24 hours after their Day 1 dose of Compound I.

As shown in Table 19, PARP activity has been inhibited 50%-93% in tumor samples taken from 6 patients in part 2 of the study, 4-6 hours after being dosed with 12 mg/m² of Compound I on Day 1, 4 or 5 of their first cycle.

TABLE 18
PARP Activity at 0.5, 4 to 6, and 24 Hours
Following Administration of Compound I on Day 1

| | | Compound I | | No. of | % Pretreatment | |
|---|---|---|---|---|---|---|
| | No. of | Starting | | Patients | PARP Activity | |
| | Patients | Dose | Time | with | Median | Range |
| Cohort | Treated | (mg/m²) | (h) | Samples | (%) | (%) |
| 1^{a} | 3 | 1 | 0.5 | 2 | 18 | 17-18 |
| | | | 4-6 | 2 | 57 | BLD-114 |
| | | | 24 | 2 | 77 | 40-114 |
| 2 | 4 | 2 | 0.5 | 3 | 10 | 9-21 |
| | | | 4-6 | 3 | 27 | 15-62 |
| | | | 24 | 2 | 36 | 22-48 |
| 3 | 3 | 4 | 0.5 | 2 | 42 | 23-60 |
| | | | 4-6 | 3 | 28 | 22-47 |
| | | | 24 | 3 | 108 | 50-264 |
| 4 | 4 | 8 | 0.5 | 3 | 1 | 1-22 |
| | | | 4-6 | 3 | 16 | 5-30 |
| | | | 24 | 3 | 9 | 7-31 |
| 5-8 | 12 | 12 | 0.5 | 12 | 14.5 | BLD-59 |
| | | | 4-6 | 12 | 13 | BLD-83 |
| | | | 24 | 12 | 29 | 3-73 |
| 9 | 6 | 18 | 0.5 | 6 | 6 | 0-21 |
| | | | 4-6 | 6 | 7.5 | 0-21 |
| | | | 24 | 6 | 14 | 0-34 |

PARP = poly (ADP-ribose) polymerase;
BLD = below limit of detection.
^{a}For the first cohort only, samples were not collected on Day 1. Data are from samples collected after the test dose of Compound I (usually Day −7).

TABLE 19
PARP Inhibition in Tumors

| | Starting | Starting | | |
|---|---|---|---|---|
| | temozolomide | Compound I | | |
| | dose | dose | Day of | % PARP |
| Patient | (mg/m²) | (mg/m²) | Biopsy | Inhibition |
| A | 100 | 4 | 1 | 79% |
| B | 135 | 12 | 1 | 91% |
| C | 135 | 12 | 4 | 85% |
| D | 135 | 12 | 4 | 50% |
| E | 170 | 12 | 5 | 93% |
| F | 170 | 12 | 1 | 77% |
| G | 170 | 12 | 1 | 98% |
| H | 200 | 12 | 1 | 90% |
| I | 200 | 12 | 1 | 88% |
| J | 200 | 18 | 1 | 98% |
| K | 200 | 18 | 1 | 94% |
| L | 200 | 18 | 1 | 89% |
| M | 200 | 18 | 1 | 86%* |
| N | 200 | 18 | 1 | 97% |

Post-treatment biopsies taken at 4-6 hours post-treatment on day indicated, except
*taken at 24 hours post-treatment on day 1 (prior to day 2 treatment).

### Example 11

### Effects in Humans: A Phase ½ Trial of the Intravenous PARP Inhibitor Compound I in Combination with the “FOLFIRI” Renimen in Patients with Advanced Colorectal Cancer who have Failed a “FOLFOX” Regimen in the First Line Metastatic Setting

A lead-in Phase 1 portion of the study identifies the dose of Compound I in combination with irinotecan, 5-FU and leucovorin to be used in a Phase 2 portion. The Phase 2 portion is an open-label multi-centre study of Compound I given in combination with FOLFIRI for patients who have received prior FOLFOX chemotherapy for 1^{st} line metastatic colorectal cancer.

The Phase 1 portion of the trial is in 2 parts. Part 1 is an open-label dose escalation study evaluating the safety and tolerability of the combination of Compound I with irinotecan (see Table 20-Part 1). Part 2 adds 5-FU +leucovorin to the combination already established in part 1 (see Table 20-Part 2). Patients are dosed in 2-week cycles to facilitate transition into the Phase 2 FOLFIRI dosing schema. Patients have histologically or cytologically proven colorectal cancer that is refractory to or who have failed FOLFOX in the first line metastatic setting, be at least 18 years of age, have good performance status (WHO 0 or 1), have adequate bone marrow, liver, and renal function as determined by routine blood tests, provide informed consent, as well as meeting several other entry criteria. Patients receive the PARP inhibitory dose of Compound I (as determined in an earlier Phase 1 study and part 1 of this trial) and FOLFIRI.

In Phase 2, Cycle numbers refers to each 2-week cycle of FOLFIRI, which is given in the standard fashion. Irinotecan (dose based upon Phase 1) is given intravenously over 90 minutes on Day 1. Leucovorin (LV 200 mg/m²) infusion begins concurrently with irinotecan, and proceeds over 2 hours on Day 1. A 5-FU bolus (400 mg/m²) and 46 hour 5-FU infusion (2400 mg/m²) immediately follows the leucovorin infusion. Compound I is added to irinotecan in escalating doses in serial patient cohorts as shown in Table 20. Compound I initially is given at a starting dose of 12 mg/m² given by 30-minute intravenous infusion 1 hour before each irinotecan dose and again 24 hours later. The starting dose of irinotecan is 150 mg/m² (about 80% of the full dose of irinotecan used in the FOLFIRI regimen). Blood samples are collected in cycle 1 to determine the PK profiles of Compound I, irinotecan, and SN-38.

Dose Limiting Toxicity (DLT) is used to determine the maximum tolerated dose (MTD) and is assessed in the first 4 weeks. Initially, 3 patients are entered into each dose level. If a DLT is observed in 1 of the first 3 patients of any cohort, an additional 3 patients are enrolled. The MTD is defined as the highest dose level at which ≦⅙ patients experience DLT during the first 4 weeks. No dose escalations of Compound I above 18 mg/m² are made. Once 6 patients treated at the MTD have completed 4 weeks, the Phase 2 portion begins. MTD is defined as a dose below that at which more than 30% (2 of up to 6 patients) of the cohort, experienced dose limiting toxicity due to the drug combination during the first 21 days of treatment. Patients who do not complete the pre-requisite time for evaluation of DLTs for any reason other than a treatment-related toxicity are replaced. The MTD is the recommended starting dose for phase 2 trials.

TABLE 20
Phase 1 doses

| | | |
|---|---|---|
| Cohort | | |
| (Dose Level) | Irinotecan Dose Level | Compound I mg/m² |

PART 1

| | | |
|---|---|---|
| −1 | 150 mg/m² | 8 mg/m² |
| 1 - Starting | 150 mg/m² | 12 mg/m² |
| dose | | |
| 2 | 180 mg/m² | 12 mg/m² |
| 3 | 180 mg/m² | 18 mg/m² |

PART 2
Use the dose identified as safe and tolerable in part-1 to combine
with 5-FU + Leucovorin

| Cohort | 5-FU dose | Leucovorin |
|---|---|---|
| 4 | A 5-FU bolus | Leucovorin (LV 200 mg/m²) |
| | (400 mg/m²) and | infusion will begin concurrently |
| | 46 hour 5-FU infusion | with irinotecan, and proceed |
| | (2400 mg/m²) | over 2 hours on Day 1. |
| | will immediately follow | |
| | the leucovorin infusion. | |

Objective response rate is the primary endpoint for the Phase 2 study. Patients have assessments for tumor response every 3 cycles of FOLFIRI. The objective response rate (RR) of the combination of Compound I with FOLFIRI is determined using Response Evaluation Criteria In Solid Tumors (RECIST) criteria. Therasse et al. New guidelines to evaluate the response to treatment in solid tumors. J Natl Cancer Inst, 2000, v. 92, pp. 205-216.

### Example 12

### Radiosensitization by the Compound of Formula 1

A contributory factor to radiation resistance in vivo is the ability of quiescent cells to repair potentially lethal damage (PLD) (Weichselbaum, R. R. and Little, J. B. The differential response of human tumors to fractionated radiation may be due to a post-irradiation repair process. Br. J. Cancer 1982; 46: 532-537). In vitro models of PLD measure the increase in survival of irradiated, growth arrested cells following delayed plating for colony formation. Recovery from potentially lethal damage was measured in vitro using LoVo cells that had been arrested in G₁ phase by growing to confluence to mimic the radio-resistant quiescent cell population in tumors. Cells were exposed to 8 Gy of γ-irradiation (Gammacel 1000 Elite, Nordian International Inc. Canada), and either harvested and plated for colony formation assay immediately or maintained as growth arrested confluent cultures for a 24-hour recovery period before harvesting and plating for the colony formation assay. Where indicated, 0.4 μM compound Formula 1 was added 30 minutes before irradiation and was present throughout the recovery incubation. As shown in Table 21, cell survival was increased approximately 7-fold following 24 hr recovery in control medium. Incubation with the compound of formula 1 during the recovery period inhibited PLD recovery by 64.9%.

TABLE 21
Inhibition of Potentially Lethal Damage Recovery (PLDR)

| | Survival at 0 | Survival at | | Inhibition |
|---|---|---|---|---|
| Treatment | time | 24 hr | % PLDR^{a} | of PLDR^{b,c} |
| 8 Gy alone | 0.03 ± 0.01 | 0.22 ± 0.026 | 694 ± 308 | |
| 8 Gy + 0.4 μM | 0.02 ± 0.01 | 0.057 ± 0.038 | 211 ± 184 | 64.9 ± 39.4 |
| the compound | | | | |
| of formula 1 | | | | |

^{a}% PLDR is calculated as 100 × (survival at 24 hr-survival at 0 time)/survival at 0 time
^{b}% inhibition of recovery is calculated as 100 − ((PLDR in presence of compound Formula 1/PLDR of control) × 100)
^{c}mean of 3 independent experiments

The in vivo efficacy of the compound of formula 1 as a radiopotentiating agent has been evaluated using two independent approaches: ex-vivo clonogenic assay and tumor growth delay analysis. For the first approach, established LoVo xenografts were treated with the compound of formula 1 (15 or 30 mg/kg; parent compound) 30 minutes prior to tumor-localized radiation at a dose of 5 Gy. 24 h later tumors were excised, disaggregated to obtain single cell suspensions and plated for colony forming assay. As shown in Table 22, the surviving fraction (SF) of tumor cells treated with the compound of formula 1 and 5 Gy was enhanced compared with radiation alone. The SF for the 15 mg/kg and 30 mg/kg IR combinations equated to that which would have been achieved using radiation doses of 8 Gy or 9.5 Gy, giving dose modification factors (DMF) of 1.6 and 1.9 respectively.

TABLE 22
Ex-vivo radiopotentiation by the Compound of Formula 1

| Treatment | CFE^{a} | SF^{b} | DMF^{c} |
|---|---|---|---|
| None | 7.6 | 1 | — |
| 5Gy | 1.3 | 0.17 | 1 |
| 15 mg/kg F1 + 5Gy | 0.44 | 0.06 | 1.6 |
| 30 mg/kg F1 + 5Gy | 0.24 | 0.03 | 1.9 |

^{a}Colony forming efficiency (% plated cells)
^{b}Surviving fraction: colony forming efficiency (CFE) as a function of that untreated control tumors
^{c}Dose modification factor: fold increase in radiation dose that would be required to give the same level of clonogenic survival as the Formula 1 plus radiation combination

For tumor growth delay studies, LoVo xenografts of approximately 250 mm³ in volume were treated with 10 Gy radiation, administered in 2 Gy fractions once daily for 5 days. In combination groups, the compound of formula 1 was administered 30 minutes prior to each 2 GY fraction at a dose of either 15 or 0.15 mg/kg (again parent compound was used). The experimental endpoint was defined to be the time required for relative tumor volume to increase to four times the volume measured at the start of treatment (RTV4). Growth delays were calculated from the difference in time taken to achieve RTV4 (days) between IR/Formula 1 treated tumors and untreated controls. As shown in Table 23, both doses of the compound of formula 1 caused a significant (36%) enhancement in the activity of radiation against LoVo xenografts.

TABLE 23
Efficacy of X-irradiation in Combination with the Compound
of Formula 1 Against LoVo Xenografts

| | | Dose | | |
|---|---|---|---|---|
| Tumor | Dose | the compound of formula | | Enhancement^{d} |
| model | IR^{a} | 1^{b} | Regimen^{c} | (%) |
| LoVo | 2 Gy | 0.15 mg/kg | Daily × 5 | 36^{e} |
| LoVo | 2 Gy | 15 mg/kg | Daily × 5 | 36^{f} |

^{a}Local tumor irradiation.
^{b}the compound of Formula 1 dosages were delivered by intraperitoneal injection.
^{c}n = 5 for all groups.
^{d}Enhancement calculated as % Enhancement = 100 × (Growth Delay with IR + Compound of Formula 1 − Growth Delay IR alone)/Growth Delay IR alone.
^{e}Significantly different from IR alone p = 0.015 (Mann-Whitney test)
^{f}Significantly different from IR alone p = 0.009 (Mann-Whitney test)

The disclosures of all cited references are incorporated herein by reference in their entirety.

We claim:

1. A dosage form for administration to a mammal, the dosage form comprising a compound of formula 1: 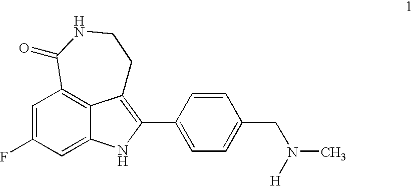
a pharmaceutically acceptable salt or solvate, or a mixture thereof, in an amount effective to provide a sustained plasma concentration value of at least 5.9 ng/mL of the compound of formula 1 for at least 24 hours after administration to the mammal.

2. The dosage form of claim 1, wherein the sustained plasma concentration value is at least 10 ng/mL.

3. The dosage form of claim 1, wherein the dosage form is a lyophilized powder for injection.

4. The dosage form of claim 1, wherein the pharmaceutically acceptable salt is a phosphate salt.

5. A dosage form for administration to a mammal, the dosage form comprising a compound of formula 1: 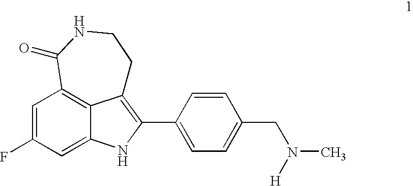
a pharmaceutically acceptable salt or solvate, or a mixture thereof, in an amount effective to inhibit a poly(ADP-ribose) polymerase enzyme by at least 50% for at least 24 hours in peripheral blood lymphocytes after administration to the mammal.

6. The dosage form of claim 5, wherein inhibition of the poly(ADP-ribose) polymerase enzyme is at least 75%.

7. The dosage form of claim 5, wherein the dosage form is a lyophilized powder for injection.

8. The dosage form of claim 5, wherein the pharmaceutically acceptable salt is a phosphate salt.

9. A dosage form for administration to a mammal, the dosage form comprising a compound of formula 1: 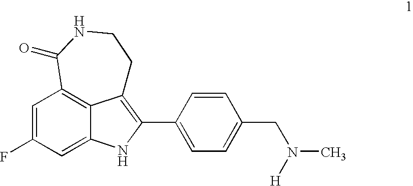
a pharmaceutically acceptable salt or solvate, or a mixture thereof, in an amount of 1-48 mg/m² expressed as free base equivalent mass of the compound of formula 1.

10. The dosage form of claim 9, wherein the amount is from 2 to 24 mg/m² expressed as free base equivalent mass of the compound of formula 1.

11. The dosage form of claim 9, wherein the amount is 12 mg/m² expressed as free base equivalent mass of the compound of formula 1.

12. The dosage form of claim 9, wherein the dosage form is a lyophilized powder for injection.

13. The dosage form of claim 9, wherein the pharmaceutically acceptable salt is a phosphate salt.

14. A dosage form for administration to a mammal, the dosage form comprising a compound of formula 1: 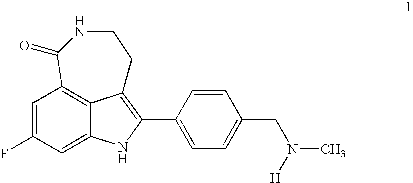
a pharmaceutically acceptable salt or solvate, or a mixture thereof, in an amount of from 2 to 96 mg expressed as free base equivalent mass of the compound of formula 1.

15. The dosage form of claim 14, wherein the amount is from 4 to 48 mg expressed as free base equivalent mass of the compound of formula 1.

16. The dosage form of claim 14, wherein the amount is 24 mg expressed as free base equivalent mass of the compound of formula 1.

17. The dosage form of claim 14, wherein the dosage form is a lyophilized powder for injection.

18. The dosage form of claim 14, wherein the pharmaceutically acceptable salt is a phosphate salt.

19. A method of treating cancer in a mammal, the method comprising administering to the mammal
(a) a compound of formula 1: 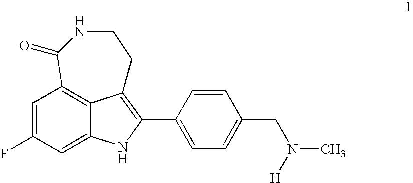
a pharmaceutically acceptable salt or solvate, or a mixture thereof in an amount effective to provide a sustained plasma concentration value of at least 5.9 ng/mL of the compound of formula 1 for at least 24 hours after administration to the mammal; and
(b) a therapeutically effective amount of at least one anti-cancer agent.

20. The method of claim 19, wherein the sustained plasma concentration value of the compound of formula 1 is at least 10 ng/mL.

21. The method of claim 19, wherein the anti-cancer agent is administrated within 1 hour after administration of the compound of formula 1.

22. The method of claim 21, wherein the anti-cancer agent is selected from the group consisting of temozolomide, irinotecan, topotecan, cisplatin, carboplatin, and doxorubicin.

23. The method of claim 22, wherein the therapeutically effective amount of temozolomide is from 100 mg/m² to 200 mg/m².

24. The method of claim 19, wherein the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

25. A kit for treating cancer in a mammal, the kit comprising:
(a) an amount of a compound of formula 1: 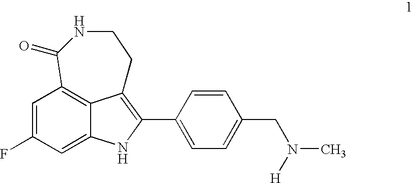
a pharmaceutically acceptable salt or solvate, or a mixture thereof, and a pharmaceutically acceptable carrier or diluent in a first unit dosage form;
(b) an amount of at least one anti-cancer agent and a pharmaceutically acceptable carrier or diluent in at least a second unit dosage form; and
(c) container for containing the first and at least the second dosage forms;
wherein the amount of the compound of formula 1 is effective to provide a sustained plasma concentration value of at least 5.9 ng/mL of the compound of formula 1 for at least 24 hours after administration to the mammal.

26. The kit of claim 25, wherein a dosage form in the first unit is a lyophilized powder for injection.

27. The method of claim 19, wherein step b) comprises administering a combination of irinotecan, 5-flourouracil and leucovorin.

28. A method of treating cancer in a mammal, the method comprising administering to the mammal
(a) a compound of formula 1: 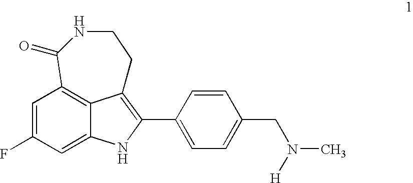
a pharmaceutically acceptable salt or solvate, or a mixture thereof in an amount effective to provide a sustained plasma concentration value of at least 5.9 ng/mL of the compound of formula 1 for at least 24 hours after administration to the mammal; and
(b) a dose of radiation effective to destroy the cancer.
